SEQUENCE LISTING (3 sequences)

SEQ ID NO: 1 - DNA, 1446 bp, unknown
  organism of the order Bacillales; proposed name Arcibacillus stibiireducens
tggcggcgtgcctaatacatgcaagtcgagcggatgtgatgaagagcttgctcttctgaacattagcggcggacgggtgagtaacacgtgggtaacctggctgtaagactgggataaccccgggaaaccggagctaataccagataataagatttctcgcatgagagatttttgaaaggtgctaaggcatcacttacaaatgggcccgcggcgcattagctagttggtggggtaaaggcctaccaaggcaacgatgcgtagccgacctgagagggtgaccggccacactggaactgagacacggtccagactcctacgggaggcagcagtagggaatcttccgcaatggacgaaagtctgacggagcaacgccgcgtgagcgaagaaggtcttcggattgtaaagctctgtctttaaggaagaacagctatgagagggaatgctcatagagtgacggtacttaaggaggaagccccggctaactacgtgccagcagccgcggtaatacgtagggggcaagcgttgtccggaattattgggcgtaaagcgcgcgcaggtggccatgtaagtctgatgtgaaattctagggctcaaccttagaactgcattggaaactgcatggcttgagagcaggagagggaagtggaattcctagtgtagcggtgaaatgcgtagatattaggaggaacaccagtggcgaaggcgacttcctggcctgttactgacactgaggcgcgaaagcgtggggagcaaacaggattagataccctggtagtccacgctgtaaacgatgagtgctatgtgttggagggtaccaccttcagtgcagtagttaacgcaataagcactccgcctggggagtacggtcgcaaggctgaaactcaaaggaattgacggggacccgcacaagcagtggagcatgtggtttaattcgaagcaacgcgaagaaccttaccaaggcttgacatccctctgaccggtgtagagatacacctttccttcgggacagaggtgacaggtggtgcatggttgtcgtcagctcgtgtcgtgagatgttgggttaagtcccgcaacgagcgcaacccctattattagttgccagcattaagttgggcactctaatgagactgccggtgacaaaccggaggaaggcggggatgacgtcaaatcatcatgccccttatgtcttgggctacacacgtgctacaatggatggtacagagggaagcgaagccgcgaggtggagccaatcccaaaaagccattctcagttcggattgtaggctgcaactcgcctacatgaagctggaattgctagtaatcgcggatcagaatgccgcggtgaatacgttcccgggtcttgtacacaccgcccgtcacaccacgagagtttgtaacacccgaagccggtggggtaacccgcaagggagctagccgtcgaaggtgg

SEQ ID NO: 2 - DNA, 20 bp, artificial
  synthetic universal bacterial oligonucleotide primer
agagtttgatcctggctcag

SEQ ID NO: 3 - DNA, 19 bp, artificial
  synthetic universal bacterial oligonucleotide primer
ggttaccttgttacgactt

(19) United States
(12) Patent Application Publication
(10) Pub. No.: US 2015/0056677 A1
(43) Pub. Date: Feb. 26, 2015

(54) MICROBES AND METHODS FOR REDUCING COMPOUNDS

(71) Applicant: University of Georgia Research Foundation, Inc., Athens, GA (US)

(72) Inventors: CHRISTOPHER A. ABIN, ATHENS, GA (US); JAMES T. HOLLIBAUGH, ATHENS, GA (US)

(21) Appl. No.: 14/466,105

(22) Filed: Aug. 22, 2014

Related U.S. Application Data
(60) Provisional application No. 61/869,422, filed on Aug. 23, 2013.

(51) Int. Cl. C12P 3/00; B09C 1/10; C02F 3/28; C02F 3/34; C12R 1/01; C12N 1/20

(52) U.S. Cl. CPC C12P 3/00; C12R 1/01; C12N 1/20; C02F 3/2813; C02F 3/34; B09C 1/10; C12N 2500/10; C02F 2103/06
USPC 435/168; 435/252.1; 210/601; 405/128.7

(57) ABSTRACT

Provided herein are biologically pure cultures of microbes having the characteristic of reducing Sb(V) to Sb(III). In one embodiment, the microbes produce Sb₂O₃ by reduction of Sb(OH)₆⁻. In one embodiment, the microbes also reduce selenite, selenate, tellurate, nitrite, nitrate, and/or arsenate. Also provided are compositions that include the microbes, and methods of using the microbes. Examples of methods include making crystalline Sb₂O₃ and converting soluble contaminants to insoluble contaminants.

### CROSS-REFERENCE TO RELATED APPLICATIONS

This application claims the benefit of U.S. Provisional Application Ser. No. 61/869,422, filed Aug. 23, 2013, which is incorporated by reference herein.

### GOVERNMENT FUNDING

The invention was made with government support under EAR-0952271, awarded by the National Science Foundation. The government has certain rights in the invention.

### BACKGROUND

At present, very little information exists on the oxidation and reduction of antimony (Sb) by living organisms (Filella et al., 2007. Earth-Science Reviews 80:195-217). Although Sb(V) is the predominant species present in oxygenated environments, numerous studies have detected thermodynamically unstable Sb(III) in aerobic freshwater, marine, and groundwater systems (Apte et al., 1986. Journal of Analytical Atomic Spectrometry 1:221-225; Cutter et al., 2001. Deep Sea Research Part II: Topical Studies in Oceanography 48:2895-2915; Cutter et al., 1991. Analytical Chemistry 63:1138-1142; Deng et al., 2001. Analytica Chimica Acta 432:293-302; Gohda, 1974. Journal of Oceanography 30:163-167; Mohammad et al., 1990. Chemical Speciation and Bioavailability 2:117-122; Sun et al., 1999. Analytica Chimica Acta 395:293-300; Takayanagi and Cossa. 1997. Water Research 31:671-674). A similar phenomenon has been observed for anaerobic environments, where oxidized species have been found to constitute as much as 50-56% of total dissolved Sb in anoxic, bottom waters of the Baltic Sea, Black Sea, and Saanich Inlet, Canada (Andreae et al., 1984. Tellus B 36B:101-117; Bertine and Lee. 1983. p. 21-38. In C. S. Wong, E. Boyle, K. W. Bruland, J. D. Burton, and E. D. Goldberg (ed.), Trace Metals in Sea Water. Plenum, New York, N.Y.; Cutter, 1991. Oceanographic Research Papers 38:S825-S843). Although a variety of abiotic mechanisms have been postulated to explain these results, some authors have implicated microbiological activity as the most likely culprit behind the presence of thermodynamically unfavorable Sb species (Andreae et al., 1984. Tellus B 36B:101-117; Bertine and Lee. 1983. p. 21-38. In C. S. Wong, E. Boyle, K. W. Bruland, J. D. Burton, and E. D. Goldberg (ed.), Trace Metals in Sea Water. Plenum, New York, N.Y.; Takayanagi and Cossa. 1997. Water Research 31:671-674). In 1972, Lyalikova and colleagues became the first to isolate and describe a microorganism capable of conserving energy from the oxidation of Sb(III) to Sb(V). Stibiobacter senarmontii, a Gram positive chemolithotrophic bacterium, was isolated from an antimony ore deposit in the former Yugoslavia and was shown to utilize oxygen as a terminal electron acceptor for the oxidation of the minerals sénarmontite (Sb₂O₃) and cervantite (Sb₂O₄) to stibiconite [Sb₃O₆(OH)] (Lyalikova, 1974. Doklady Akademii Nauk SSSR 205:1228-1229; Lyalikova, 1974. Mikrobiologiia 43:941-948; Lyalikova, et al., 1976. Mikrobiologiia 45:552-554). Although the authors described numerous morphological characteristics, such as its rod-like shape, three polar flagella, and electron-dense cytoplasmic inclusions, no attempts were made to identify the genes or enzymes responsible for Sb(III) oxidation. Unfortunately, the isolate was never preserved in an international culture collection or biorepository, thus eliminating any possibility for further study of its novel biochemistry.

Dissimilatory metal-reducing bacteria (DMRB) are a phylogenetically diverse group of microorganisms capable of conserving energy to support growth by coupling the oxidation of organic acids, alcohols, aromatics, or H₂ to the reduction of metals and metalloids as terminal electron acceptors (Lovley, 1993. Annual Review of Microbiology 47:263-290). Although the list of DMRB continues to grow as more extreme and unusual environments are examined, the most highly studied examples include Gram-negative members of the β, γ, δ, and ε subgroups of the Proteobacteria and Gram-positives of the Phylum Firmicutes. Since the isolation and characterization of the first iron- and manganese-reducing bacteria in the late 1980's, an impressive array of metals and radionuclides have since been found to serve as terminal oxidants for DMRB, including Fe(III), Mn(IV), V(V), U(VI), Te(VI), Te(IV), Se(VI), Se(IV), As(V), Cr(VI), Co(III), Tc(VII), Np(V), and Pu(VI). DMRB-mediated reduction has been regarded as an important process controlling the fate of toxic metals and radionuclides in anaerobic environments such as sediments, submerged soils, and groundwaters (Han and Ji-Dong. 2010. p. 153-176. In R. Mitchell and G. Ji-Dong (ed.), Environmental Microbiology. Jon Wiley and Sons Ltd., Hoboken, N.J.; McMahon and Chapelle. 2008. Ground Water 46:259-271; Nealson et al., 2002. Antonie van Leeuwenhoek 81:215-222). Owing to their low solubility, the reduced species of many metals and radionuclides precipitate from aqueous solution as immobile oxide, sulfide, and/or carbonate minerals during the microbial reduction process (Gorby and Lovley. 1992. Environmental Science & Technology 26:205-207; Liu et al., 2002. Biotechnology and Bioengineering 80:637-649; Lloyd et al., 2001. Hydrometallurgy 59:327-337). Thus, DMRB-mediated metal reduction represents a potentially simple, sustainable, and cost-effective bioremediation strategy for the in-situ immobilization and containment of hazardous metals and radionuclides in contaminated aquatic systems and subsurface environments. Through their metabolism, DMRB not only mediate the transformation of toxic metals to more innocuous forms, but they intimately link those processes with the global biogeochemical cycling of carbon.

### SUMMARY OF THE APPLICATION

Provided herein is a biologically pure culture of a microbe that has the characteristic of reducing Sb(V) to Sb(III). In one embodiment, the microbe produces Sb₂O₃ by reduction of Sb(OH)₆⁻. In one embodiment, the Sb₂O₃ is sénarmontite, valentinite, or a combination thereof. In one embodiment, the microbe also has the characteristics of reducing a compound selected from selenite, selenate, tellurate, nitrite, nitrate, arsenate, DMSO, and a combination thereof. In one embodiment, the microbe has the characteristic of not reducing a compound selected from sulfate, sulfite, thiosulfate, tetrathionate, elemental sulfur, chromate, metavanadate, iron (III) hydroxide (insoluble), iron (III)-EDTA (soluble), manganese dioxide, molybdate, chlorate, perchlorate, and a combination thereof. In one embodiment, the microbe has a 16S rRNA coding region having at least 97% identity to SEQ ID NO:1. The microbe may be, and in one embodiment is, a member of the order Bacillales. In one embodiment, the microbe has the characteristics of MLFW-2 as deposited with the American Type Culture Collection under number PTA-120556 in accordance with the provisions of the Budapest Treaty.

Also provided is a composition that includes the microbe described herein and a substrate, wherein the substrate is may be selenite, selenate, tellurate, nitrite, nitrate, arsenate, a compound that includes Sb(V), or a combination thereof. The composition may also include a molecule that is oxidized in conjunction with the reduction of the substrate. In one embodiment, the molecule is an organic molecule, such as lactate, pyruvate, fumarate, and formate, casamino acids, yeast extract, and a combination thereof. In one embodiment, the substrate is a compound that includes Sb(V), such as Sb(OH)₆⁻, and the molecule that is oxidized has a standard midpoint reduction potential (E°′) of less than +94 mV. In one embodiment, the composition includes an environmental sample. The environmental sample may include selenite, selenate, tellurate, nitrite, nitrate, arsenate, a compound that includes Sb(V), or a combination thereof. Examples of environmental samples include, but are not limited to, soil, groundwater, surface water, wastewater, sediment, or a combination thereof.

Also provided are methods. In one embodiment, a method includes culturing microbe described herein in a composition including a substrate that is reduced by the microbe under suitable conditions. In one embodiment, the substrate is a compound that includes Sb(V), such as Sb(OH)₆⁻. In those embodiments where the reduction produces Sb₂O₃, the Sb₂O₃ is sénarmontite, valentinite, or a combination thereof. The method may further include isolating the sénarmontite, the valentinite, or the combination thereof. In one embodiment, the substrate may be selenite, selenate, tellurate, nitrite, nitrate, arsenate, or a combination thereof. In one embodiment, the composition includes an environmental sample, such as soil, groundwater, surface water, sediment, or a combination thereof, or a waste stream.

Further provided is a method for making crystalline Sb₂O₃. The method includes culturing a microbe described herein in a composition. The composition includes a compound that includes Sb(V), wherein the reduction of the Sb(V) results in Sb₂O₃. The Sb₂O₃ produced is sénarmontite, valentinite, or a combination thereof. In one embodiment, the culturing is under conditions suitable for the precipitation of the Sb₂O₃. Optionally, the sénarmontite, the valentinite, or the combination thereof may be isolated.

Also provided herein is a method that includes culturing a microbe described herein in a sample that includes a soluble contaminant that is reduced by the microbe under suitable conditions. The soluble contaminant may be selenite, selenate, tellurate, nitrite, nitrate, arsenate, a compound that includes Sb(V), or a combination thereof. In one embodiment, the reduction converts the contaminant from a soluble form to an insoluble form. The sample may be an environmental sample, such as one that includes soil, groundwater, surface water, waste water, sediment, or a combination thereof. In one embodiment, the sample may include a waste stream.

Also provided herein is a method that includes adding a microbe described herein to an environment that includes a soluble contaminant that is reduced by the microbe under suitable conditions. The soluble contaminant may be selenite, selenate, tellurate, nitrite, nitrate, arsenate, a compound that includes Sb(V), or a combination thereof, wherein the environment is anoxic. In one embodiment, the reduction converts the contaminant from a soluble form to an insoluble form. The environment may be one that includes soil, groundwater, surface water, waste water, sediment, a waste stream or a combination thereof.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Morphology of strain MLFW-2. Scanning electron micrograph of cells of strain MLFW-2 grown on antimonate and lactate in a basal salts medium.

FIG. 2. Anaerobic growth of strain MLFW-2 at 30° C. with 2 mM antimonate and 1 mM lactate as electron acceptor and donor, respectively. Symbols are as follows: closed diamonds, antimonate; open diamonds, antimonite; closed triangles, lactate; open triangles, acetate; closed circles, cells. The dashed line shows the antimonate concentration in a control lacking lactate. The dotted line shows the cell density in a control containing lactate but not antimonate.

FIG. 3. Scanning electron micrographs (A and B) of the microcrystals produced by strain MLFW-2 during DSbR in basal salts medium. X-ray diffraction patterns of (C) standard Sb₂O₃ and (D) the microcrystals produced by strain MLFW-2. Peaks associated with cubic (JCPDS05-0534) and orthorhombic (JCPDS 11-689) Sb₂O₃ in (C) are capped by squares and circles, respectively.

FIG. 4. EDS spectra of the cubic (A) and “bowtie”-shaped (B) microcrystals produced by strain MLFW-2 during DSbR, as well as standard Sb₂O₃ of >99.9% purity (C). The insets show the particular areas analyzed. “N” refers to the total number of random fields or individual microcrystals analyzed, while “SD” corresponds to the standard deviation associated with the average atomic percentages obtained for Sb and O.

FIG. 5. Phylogenetic position of strain MLFW-2 within the order Bacillales based on 16S rRNA gene sequence analysis. Tree topology and evolutionary distances were obtained by the neighbor-joining method with Jukes-Cantor distances. Numbers at the nodes give bootstrap support for the node as a percentage of 1,000 replicates. Scale bar gives substitutions per base pair.

FIG. 6. (A) UV-vis absorption spectrum and (B) photoluminescence spectrum of the bulk precipitate of Sb₂O₃ produced by strain MLFW-2.

FIG. 7. Micrographs of strain MLFW-2^{T}. (A) Scanning electron micrograph of a culture grown in BSM-1 using lactate and antimonate as the electron donor and acceptor, respectively. Scale bar, 1 μm. (B) Transmission electron micrograph of negatively stained cells grown in BSM-5 using lactate and arsenate. Scale bar, 1 μm. (C) Transmission electron micrograph of an ultrathin section of cells grown under the same conditions as in (B). Scale bar, 500 nm. (D) Magnified section of the cell envelope. Scale bar, 40 nm.

FIG. 8. Neighbor-joining phylogenetic tree based on 16S rRNA gene sequences showing the relationship between strain MLFW-2^{T} and type strains of species of the order Bacillales. Evolutionary distances were generated using the model of Jukes and Cantor (1969, Evolution of protein molecules. In Mammalian Protein Metabolism, vol. 3, pp. 21-132. Edited by H. N. Munro. New York: Academic Press) based on the pairwise comparison of approximately 1,440 unambiguously aligned nucleotides. Bootstrap values (≧70%) based on 1,000 resamplings are shown at the nodes. Thermicanus aegyptius ET-5b^{T} was used as an outgroup. GenBank accession numbers are given in parentheses. Bar, 0.02 substitutions per nucleotide position.

FIG. 9. Maximum-parsimony phylogenetic tree based on partial 16S rRNA gene sequences showing the relationship between Arcibacillus stibiireducens sp. nov. MLFW-2^{T} and type strains of species of the order Bacillales. Bootstrap values are expressed as percentages of 1,000 replications; only values ≧70% are shown at branch nodes. Thermicanus aegyptius ET-5b^{T} was used as an outgroup. Bar, 60 substitutions.

FIG. 10. Maximum-likelihood phylogenetic tree based on partial 16S rRNA gene sequences showing the relationship between Arcibacillus stibiireducens sp. nov. MLFW-2^{T} and type strains of species of the order Bacillales. Bootstrap values are expressed as percentages of 1,000 replications; only values ≧70% are shown at branch nodes. Thermicanus aegyptius ET-5b^{T} was used as an outgroup. Bar, 0.03 substitutions per nucleotide position.

FIG. 11. Two-dimensional thin-layer chromatogram of polar lipids of strain MLFW-2^{T}. PG, phosphatidylglycerol; PE, phosphatidylethanolamine; PL, unknown phospholipid; L, unknown polar lipid.

### DETAILED DESCRIPTION OF ILLUSTRATIVE EMBODIMENTS

Provided herein are microbes having the characteristic of reducing a substrate that includes Sb(V), such as Sb(OH)₆⁻ (also referred to as antimonate), coupled to the oxidation of a compound. In one embodiment, the ultimate product of the reduction is the cubic form of antimony trioxide, Sb₂O₃, also referred to as senarmontite, the orthorhombic form of antimony trioxide, β-Sb₂O₃, also referred to as valentinite, or the combination thereof. In one embodiment, the product valentinite occurs in conditions where reduced sulfur is present at a concentration of up to 150 uM. In conditions that include reduced sulfur at concentrations increasing from 150 uM Sb₂₅₃ may be formed in addition to Sb₂O₃.

In one embodiment, such a microbe also has the characteristic of reducing other substrates coupled to the oxidation of a compound. Examples of other substrates that are reduced by a microbe described herein, and the resulting products, are shown on Table 1.

TABLE 1

| Substrate | Product |
|---|---|
| Selenite or Selenate | Elemental selenium [Se(0)] |
| tellurate¹ | Elemental tellurium [Te(0)] |
| Nitrite or Nitrate | Ammonium |
| Arsenate | Arsenite |
| Dimethylsulfoxide (DMSO) | Dimethylsulfide (DMS) |

¹During tellurate respiration, tellurite is transiently formed as an intermediate, but is quickly reduced to form a black-colored precipitate of elemental tellurium [Te(0)].

In one embodiment, such a microbe will not use the following compounds as substrates for reduction: Sulfate, Sulfite, Thiosulfate, Tetrathionate, Elemental sulfur, Chromate, Metavanadate, Iron (III) hydroxide (insoluble), Iron (III)-EDTA (soluble), Manganese dioxide, Molybdate, Chlorate, or Perchlorate.

In one embodiment, a microbe having the characteristic of reducing a substrate that includes Sb(V), and optionally one or more of the compounds of Table 1, and is a member of the order Bacillales. As described in Example 2, a microbe having the characteristic of reducing a substrate that includes Sb(V), and optionally one or more of the compounds of Table 1, is not classified as a known species, and the name Arcibacillus stibiireducens gen. nov., sp. nov. is proposed.

In one embodiment, such a microbe includes a 16S rRNA coding region that has at least 90%, at least 91%, at least 92%, at least 93%, at least 94%, at least 95%, at least 96%, at least 97%, at least 97.1%, at least 97.2%, at least 97.3%, at least 97.4%, at least 97.5%, at least 97.6%, at least 97.7%, at least 97.8%, at least 97.9%, at least 98%, at least 98.1%, at least 98.2%, at least 98.3%, at least 98.4%, at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, at least 99.9%, identity, or 100% identity, to the sequence of SEQ ID NO:1 (also available as Genbank accession number KF387535). Two nucleotide sequences may be compared using the BESTFIT algorithm in the GCG package (version 10.2, Madison Wis.), or the Blastn program of the BLAST 2 search algorithm, as described by Tatusova, et al. (FEMS Microbiol Lett 1999, 174:247-250), and available through the World Wide Web, for instance at the internet site maintained by the National Center for Biotechnology Information, National Institutes of Health. In one embodiment, the default values for all BLAST 2 search parameters are used.

SEQ ID NO: 1
TGGCGGCGTGCCTAATACATGCAAGTCGAGCGGATGTGATGAAGAGCTTG
CTCTTCTGAACATTAGCGGCGGACGGGTGAGTAACACGTGGGTAACCTGG
CTGTAAGACTGGGATAACCCCGGGAAACCGGAGCTAATACCAGATAATAA
GATTTCTCGCATGAGAGATTTTTGAAAGGTGCTAAGGCATCACTTACAAA
TGGGCCCGCGGCGCATTAGCTAGTTGGTGGGGTAAAGGCCTACCAAGGCA
ACGATGCGTAGCCGACCTGAGAGGGTGACCGGCCACACTGGAACTGAGAC
ACGGTCCAGACTCCTACGGGAGGCAGCAGTAGGGAATCTTCCGCAATGGA
CGAAAGTCTGACGGAGCAACGCCGCGTGAGCGAAGAAGGTCTTCGGATTG
TAAAGCTCTGTCTTTAAGGAAGAACAGCTATGAGAGGGAATGCTCATAGA
GTGACGGTACTTAAGGAGGAAGCCCCGGCTAACTACGTGCCAGCAGCCGC
GGTAATACGTAGGGGGCAAGCGTTGTCCGGAATTATTGGGCGTAAAGCGC
GCGCAGGTGGCCATGTAAGTCTGATGTGAAATTCTAGGGCTCAACCTTAG
AACTGCATTGGAAACTGCATGGCTTGAGAGCAGGAGAGGGAAGTGGAATT
CCTAGTGTAGCGGTGAAATGCGTAGATATTAGGAGGAACACCAGTGGCGA
AGGCGACTTCCTGGCCTGTTACTGACACTGAGGCGCGAAAGCGTGGGGAG
CAAACAGGATTAGATACCCTGGTAGTCCACGCTGTAAACGATGAGTGCTA
TGTGTTGGAGGGTACCACCTTCAGTGCAGTAGTTAACGCAATAAGCACTC
CGCCTGGGGAGTACGGTCGCAAGGCTGAAACTCAAAGGAATTGACGGGGA
CCCGCACAAGCAGTGGAGCATGTGGTTTAATTCGAAGCAACGCGAAGAAC
CTTACCAAGGCTTGACATCCCTCTGACCGGTGTAGAGATACACCTTTCCT
TCGGGACAGAGGTGACAGGTGGTGCATGGTTGTCGTCAGCTCGTGTCGTG
AGATGTTGGGTTAAGTCCCGCAACGAGCGCAACCCCTATTATTAGTTGCC
AGCATTAAGTTGGGCACTCTAATGAGACTGCCGGTGACAAACCGGAGGAA
GGCGGGGATGACGTCAAATCATCATGCCCCTTATGTCTTGGGCTACACAC
GTGCTACAATGGATGGTACAGAGGGAAGCGAAGCCGCGAGGTGGAGCCAA
TCCCAAAAAGCCATTCTCAGTTCGGATTGTAGGCTGCAACTCGCCTACAT
GAAGCTGGAATTGCTAGTAATCGCGGATCAGAATGCCGCGGTGAATACGT
TCCCGGGTCTTGTACACACCGCCCGTCACACCACGAGAGTTTGTAACACC
CGAAGCCGGTGGGGTAACCCGCAAGGGAGCTAGCCGTCGAAGGTGG

In one embodiment, a microbe having the characteristic of reducing a substrate that includes Sb(V), and optionally one or more of the compounds of Table 1, is an obligate anaerobe. For instance, a microbe having the characteristic of reducing a substrate that includes Sb(V), and optionally one or more of the compounds of Table 1, does not grow in the presence of air (21% O₂), or 2% O₂.

In one embodiment, the microbe is MLFW-2 (also referred to herein as MLFW-2^{T}). The microbe MLFW-2 was deposited with American Type Culture Collection (ATCC), 10801 University Boulevard, Manassas, Va., 20110-2209, USA, on Sep. 25, 2013, and assigned Accession Number PTA-120556. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

A microbe having the characteristic of reducing a substrate that includes Sb(V), and optionally one or more of the compounds of Table 1, may be isolated from different environments. Examples of environments include, for instance, anoxic bottom waters of lakes and anoxic sediments of lakes. Examples of lakes include, but are not limited to, those having a high pH of 9 and above, e.g., soda lakes or alkaline lakes; however, a suitable environment may have a pH lower than 9. Other environments include anoxic sediments that contain elevated levels of antimony, such as mines (e.g., stibnite mines), geothermal springs, and sites naturally or artificially contaminated with, for instance, antimony (e.g., antimonate), or any of the substrates in Table 1. In one embodiment, since a microbe described herein may form spores, it may be isolated from aerobic environments as a spore.

A microbe described herein may be present in a biologically pure culture. As used herein, as “biologically pure culture” refers to a culture in which the microbe is the sole replicating cell present. A “biologically pure culture” may, and typically does, include other inert components such as nutrients that can be used by the microbe for replication, including substrates that can be reduced by the microbe. As used herein, a “composition” containing a microbe described herein refers to a mixture to which the microbe has been added. Thus, for instance, a “composition” includes a mixture that has been modified by the addition of a microbe described herein. Examples of mixtures include, but are not limited to, a sample that has been removed from the environment, and a sample that is present in the environment. In one embodiment, a “composition” does not include an environmental sample that has not been further modified by addition of a microbe described herein.

Also provided are methods for using the microbes described herein. In one embodiment, the method includes culturing the microbe in a medium that includes a substrate under conditions suitable for the reduction of the substrate. Substrates can include, for instance, oxyanions, including, but not limited to, oxyanions of selenium (for instance, selenate and selenite) oxyanions of tellurium (for instance, tellurate), oxyanions of nitrogen (for instance, nitrate and nitrite), oxyanions of arsenic (for instance, arsenate), and antimonate. Other substrates include, but are not limited to, compounds that include antimony in the +5 oxidation state [Sb(V)], such as Sb(OH)₆⁻. Conditions that are “suitable” for an event to occur, such as the reduction of Sb(OH)₆⁻, or “suitable” conditions are conditions that do not prevent such events from occurring. Thus, these conditions permit, enhance, facilitate, and/or are conducive to the event. Conditions suitable for the reduction of a substrate by a microbe described herein include, for instance,

The culturing also includes a molecule that can be oxidized by the microbe. Examples of such molecules include, but are not limited to, simple organic molecules such as lactate, pyruvate, fumarate, and formate, casamino acids, and yeast extract, and inorganic molecules. Examples of inorganic molecules include H₂. In one embodiment, any redox couple (organic or inorganic) for which the standard midpoint reduction potential (E°′) is lower than that of the Sb(V)/Sb(III) redox couple (E°′=+94 mV) can serve as an electron donor for the reduction of Sb(V) to Sb(III). The E°′ of a redox half reaction is measured under standard state conditions (1 atm pressure, 25° C., pH 7, and all reactants and products dissolved at 1 molar concentration in water).

The culturing includes incubation with any nutrients necessary for viability of the microbe. In one embodiment, such as culturing a microbe having the characteristics of MLFW-2, an example of culturing conditions includes salts such as K₂CO₃, KH₂PO₄, K₂HPO₄, NH₄Cl: vitamins such as 4-aminobenzoic acid, D-biotin, folic acid, pyridoxine-HCl, riboflavin, thiamine-HCl, nicotinic acid, D-pantothenic acid, α-lipoic acid, and vitamin B12; and trace metals such as FeCl₂, MgCl₂, CaCl₂, ZnCl₂, MnCl₂, H₃BO₃, CoCl₂, CuCl₂, NiCl₂, and Na₂MoO₄. The medium may be at a pH of between 7 and 10, such as between 8.2 and 8.5, and a salinity of between 0 and 5% NaCl, such as 0.75% NaCl. The culturing conditions may include reduced sulfur (e.g., 100 μM) in the form of Na₂S to act as a sulfur source and reducing agent to poise the redox potential of the medium low enough to allow for growth. In one embodiment, the culturing conditions are anaerobic.

In one embodiment, the method includes the production of antimony trioxide, Sb₂O₃. The antimony trioxide produced may be the cubic form of antimony trioxide, also referred to as senarmontite, the orthorhombic form of antimony trioxide, β-Sb₂O₃, also referred to as valentinite, or the combination thereof. The method includes culturing under suitable conditions a microbe described herein with a substrate that includes Sb(V), such as Sb(OH)₆⁻ and a molecule that can be oxidized. In one embodiment, the culturing conditions do not include detectable amounts of reduced sulfur. In one embodiment, the culturing conditions include reduced sulfur at a concentration of between 100 and 150 μM, and may be in the form of Na₂S. Without intending to be limited by theory, the Na₂S may act as a reducing agent and sulfur source for the microbe. Optionally, the orthorhombic form of antimony trioxide precipitates as a bowtie-shaped Sb₂O₃ crystal, and/or the cubic form of antimony trioxide precipitates as a cube-shaped Sb₂O₃ crystal. The crystalline antimony trioxide may be isolated from the cells and the other components of the culture by, for instance, one or more rounds of centrifugation and washing. In one embodiment, the cubic and orthorhombic forms of antimony trioxide may be separated. The antimony trioxide may be used in nanotechnology, optics, and/or electronics industries. For instance, antimony trioxide may serve as a catalyst for the production of polyethylene terephthalate (PET) plastics used for food packaging; as a high efficiency fire retardant in plastics, paints, adhesives, and textiles; as a fining and covering agent for rubber, ceramics, enamels, fabrics, and fiber products; as a catalyst in organic synthesis; as a clarifying agent in optical glass and cathode ray tubes; as a white pigment in paints; as a component of fast-response pH electrodes; and/or as an anodic component of lithium-ion batteries.

In one embodiment, the method includes the production of Sb₂S₃. The method includes culturing under suitable conditions a microbe described herein with a substrate that includes Sb(V), such as Sb(OH)₆⁻ and a molecules that can be oxidized. In one embodiment, the culturing conditions include reduced sulfur, for instance, in excess of 150 μM.

In one embodiment, the method includes the production of elemental selenium [Se(0)] or tellurium [Te(0)]. The method includes culturing under suitable conditions a microbe described herein with a substrate that includes an oxyanion of selenium (for instance, selenite or selenate) or an oxyanion of tellurium (for instance, tellurate), and a molecule that can be oxidized.

In one embodiment, the method includes the production of ammonium. The method includes culturing under suitable conditions a microbe described herein with a substrate that includes an oxyanion of nitrogen (for instance, nitrite or nitrate), and a molecule that can be oxidized. The production of ammonium from an oxyanion of nitrogen is referred to as dissimilatory nitrate or nitrite reduction to ammonium (DNRA), a process that can act as a sink for fixed nitrogen in anoxic environments by preventing its gaseous escape as N₂O, NO, and/or N₂ through conventional denitrification pathways.

In one embodiment, the method includes the reduction of arsenate to arsenite. The method includes culturing under suitable conditions a microbe described herein with a substrate that includes arsenate and a molecule that can be oxidized.

In one embodiment, the method includes remediation of a sample. The remediation may be decreasing the concentration of a contaminant in a sample, or converting a contaminant in a sample to a different form. The different form may have decreased toxicity, decreased solubility in a solvent such as an aqueous solvent, and/or decreased bioavailability. In one embodiment, decreasing the concentration includes removal of the contaminant from the sample. The method includes culturing a microbe described herein with a sample under suitable conditions for reduction of the contaminant. The sample may be one that has been removed from the environment, or may be present in the environment. The environment may include soil, groundwater, surface water, sediment, or a combination thereof. In one embodiment, the sample may include an industrial waste stream. The sample includes, or may be supplemented to include, a substrate that can be reduced by a microbe described herein. The contaminant may be one that includes Sb(V), selenite, selenate, tellurate, nitrite, nitrate, and/or arsenate.

In one embodiment, the reduction of the contaminant is the conversion of the contaminant from a soluble form to an insoluble form. This can decrease the environmental mobility of the metal and provide a method of recovering a metal from the waste stream where it might otherwise be lost. For instance, in an embodiment where the contaminant is antimony and is present as the soluble Sb(OH)₆⁻, the method results in immobilization of the antimony by converting it to an insoluble form, Sb₂O₃. Insoluble material may be collected on filters or in settling ponds, for example, or on the walls of the reaction chamber. In one embodiment, the environmental sample in the method of remediation does not include a detectable level of a chelating agent.

Also provided herein is a method for isolating a microbe having the characteristic of reducing a substrate that includes Sb(V), such as Sb(OH₆⁻), coupled to the oxidation of a compound. The method includes culturing a microbe with any nutrients necessary for viability of the microbe and a concentration of reduced sulfur that is at a concentration of, at least, or no greater than, 50 μM, 100 μM, 150 μM, 200 μM, or 250 μM. In one embodiment, the reduced sulfur may be in the form of Na₂S. In one embodiment, the method includes repeated sub-culturing to enrich for a microbe having the characteristic of reducing a substrate that includes Sb(V), such as Sb(OH₆⁻), coupled to the oxidation of a compound.

The present invention is illustrated by the following examples. It is to be understood that the particular examples, materials, amounts, and procedures are to be interpreted broadly in accordance with the scope and spirit of the invention as set forth herein.

### Example 1

### Dissimilatory Antimonate Reduction and Production of Antimony Trioxide Microcrystals by a Novel Microorganism

This example provides the first unequivocal evidence that a bacterium is capable of conserving energy for growth and reproduction from the reduction of antimonate. Moreover, microbiological antimonate reduction may serve as a novel route for the production of antimony trioxide microcrystals of commercial significance.

Antimony (Sb) is a toxic metalloid that belongs to Group 15 of the periodic table along with nitrogen (N), phosphorus (P), arsenic (As), and bismuth (Bi). Sb can exist in a variety of oxidation states (−3, 0, 3, and 5) in nature, but is most commonly found in either the +3 or +5 states. In aqueous solution at neutral pH, Sb(III) and Sb(V) do not exist as free ions—instead they undergo hydrolysis to form Sb(OH)₃⁰ (“antimonite”) and Sb(OH)₆⁻ (“antimonate”), respectively (Filella et al., 2002, Earth-Sci.Rev., 59:265-285). Under oxic to slightly reducing conditions, antimonate is the thermodynamically favored form, while antimonite is predicted to predominate only under anoxic conditions. Sb is a strongly chalcophilic element and most often occurs as the mineral stibnite (Sb₂S₃) or in close association with sulfide-bearing ores of Cu, Pb, Au, Ag, and As (Filella et al., 2002, Earth-Sci. Rev., 57:125-176). Several oxygen-bearing mineral phases such as cervantite (Sb₂O₄), stibiconite [Sb₃O₆(OH)], sénarmontite (cubic Sb₂O₃), and valentinite (orthorhombic Sb₂O₃) are the principal weathering products of stibnite (Biver et al., 2013, Geochim. Cosmochim. Acta, 109:268-279). Sb and its compounds have been classified as pollutants of priority interest by the U.S. Environmental Protection Agency (USEPA. Water related fate of the 129 priority pollutants; EP-440/4-79-029A; United States Environmental Protection Agency: Washington, D.C., 1979) and European Union (CEC. Council directive 76/464/EEC of 4 May 1976 on pollution caused by certain dangerous substances discharged into the aquatic environment of the community. OJEC L-129, 23-29; Council of the European Communities Brussels, Belgium, 1976) for the past four decades.

Antimony is currently the ninth-most mined metal worldwide (Scheinost et al., 2006, Geochim. Cosmochim. Acta, 70:3299-3312), with commercial importance to a diverse array of industries. For example, it is used in the semiconductor industry as a doping agent in the manufacture of infrared detectors, diodes, and Hall-effect devices (Filella et al., 2002, Earth-Sci. Rev., 57:125-176). It also forms alloys that are exploited in the manufacture of lead-acid automobile batteries, type-metal for printing presses, small arms bullets, cable sheathing, and solders. A compound of great commercial interest is antimony trioxide (Sb₂O₃). It is used as a flame retardant, pigment, ceramic opacifier, glass decolorizing agent, mordant, PVC stabilizer, and catalyst for the production of polyethyleneterephthalate (PET) (Filella et al., 2002, Earth-Sci. Rev., 57:125-176). Compounds such as potassium antimonyl tartrate and pentavalent antimonials have historically been used to treat a variety of digestive ailments and tropical protozoan diseases such as leishmaniasis (Frézard et al., 2009. Molecules, 14:2317-2336).

Although a great deal of literature exists concerning the interactions between microorganisms and metalloids such as As (van L is et al., 2013, Biochim. Biophys. Acta Bioenerg, 1827:176-188) and Se (Stolz et al., 2006, Ann. Rev. Microbiol., 60:107-130), microbe-Sb interactions remain poorly understood. Antimonite has been shown to be unstable in the presence of oxygen, yet several studies have detected it in aerobic freshwater (Sun et al., 1993, Anal. Chim. Acta, 276:33-37, Cutter et al., 1991, Anal. Chem., 63:1138-1142) marine (Cutter et al., 1991, Anal. Chem., 63:1138-1142, Sun et al., 1999. Anal. Chim. Acta, 395: 293-300), and groundwater (Willis et al., 2011, Aquat. Geochem., 17:775-807) systems. Similarly, antimonate has been found to constitute as much as 50% of total dissolved Sb in the anoxic bottom waters of the Baltic (Andreae et al., 1984, Tellus B, 36:101-117) and Black Seas (Cutter, 1991, Oceanogr. Res. Papers, 38:S825-S843). While a variety of abiotic mechanisms have been put forth to account for these observations, biological activity remains as a plausible explanation for the geochemical disequilibria. The only microorganism shown to conserve energy from antimonite oxidation is the chemoautotrophic bacterium Stibiobacter senarmontii (Lyalikova, 1974, Mikrobiol., 43:941-948). The capacity to oxidize antimonite has also been documented for a strain of Agrobacterium tumefaciens as well as a species of eukaryotic alga belonging to the order Cyanidiales, but it was not clear if these processes were tied to energy conservation (Lehr et al., 2007, Appl. Environ. Microbiol., 73:2386-2389).

To our knowledge, microbial dissimilatory reduction of antimonate (DSbR) has not yet been demonstrated. The antimonate/antimonite redox couple has a favorable standard midpoint potential (E°′) of +94 mV at neutral pH (Diemar et al., 2009, Pure Appl. Chem., 81:1547-1553, Wilson et al., 2010, Environ. Pollut., 158:1169-1181), potentially allowing microorganisms to derive useable energy from the reduction of antimonate coupled to the oxidation of a variety of organic and inorganic compounds. Evidence for antimonate reduction has been obtained from experiments with Sb-rich, anoxic sediments collected from a defunct stibnite mine near Yellow Pine, Id., USA. When these sediments were amended with 1 mM antimonate, incubation under anaerobic conditions resulted in the complete reduction of antimonate to antimonite within 100 hours. Absence of antimonate reduction in heat-killed controls suggested that this process was not governed by abiotic reactions. Hockmann et al., 2013, In: 12^{th} International Conference on the Biogeochemistry of Trace Elements (ISTEB, Athens, Ga.)) have also demonstrated that incubation of Sb-contaminated soils under waterlogged (and presumably anoxic) conditions resulted in the rapid conversion of endogenous antimonate to antimonite.

Reported here is the enrichment and isolation of a bacterium capable of respiring antimonate as a terminal electron acceptor (TEA) for anaerobic respiration, producing crystalline Sb₂O₃ as a by-product. This discovery not only establishes a definitive link between microorganisms and the reductive side of the Sb biogeochemical cycle, but may provide a novel route for the synthesis of Sb₂O₃ microcrystals potentially suitable for commercial applications.

### Materials and Methods

Study Site and Sample Collection. The site of sample collection was located at Navy Beach, on the southwestern shore of Mono Lake, Calif., USA. Mono Lake is an alkaline, hypersaline lake that occupies a hydrographically closed and volcanically active basin on the eastern edge of the Sierra Nevada mountain range (Bischoff et al., 1993, Geochim. Cosmochim. Acta., 57:3855-3865). Shoreline springs are prominent along the northwestern, western, and southwestern shores of the lake, where they occur as either diffuse seepages from littoral sands or as artesian features with clearly defined orifices at or below lake level (Basham et al., 1988, Analyses of the shoreline springs in the Mono Basin, Calif.: with applications to the groundwater system. M.S. Thesis, University of California, Santa Cruz, Calif., 1988; Lee, K., 1969, Infrared exploration for shoreline springs at Mono Lake, Calif. Ph.D. Dissertation, Stanford University, Palo Alto, Calif.). The Navy Beach Warm Springs (NBWS) site consists of three interconnected geothermal springs that occupy a 150 m trend oriented from SW to NE relative to the shoreline at Navy Beach. Previous studies have demonstrated that the waters issuing from these springs are deeply circulating groundwaters that do not originate from within the lake and have a radiocarbon age in excess of 22,000 years (Neumann et al., 1995, Water Resour. Res., 31:3183-3193, Oremland et al., 1987, Geochim. Cosmochim. Acta., 51:2915-2929). The temperature and pH of the NBWS waters have remained fairly constant at 35.2±3.4° C. and 6.6±0.2, respectively, since sampling began in 196 (Bischoff et al., 1993, Geochim. Cosmochim. Acta., 57:3855-3865).

Anoxic sediments were collected by extracting a 35 cm core from an area receiving hydrological inputs from NBWS-1, the northernmost feature of the NBWS closest to the shoreline of Mono Lake (37° 56′28.7″ N, 119°1′22.4″ W). The core was sectioned aseptically at intervals of approximately 5 cm and sediment subsamples (200 mL) were stored in sealed mason jars and maintained at 4° C. during transport to the University of Georgia (UGA). The total metal content of a section of the core from 5-10 cm depth was determined following acid digestion according to previously published methods (Imperato et al., 2003, Environ. Pollut., 124:247-256). Whole sediment was dried in an oven overnight at 75° C. and a 5 g subsample was added to 100 mL of 3:1 concentrated HCl:HNO₃ and refluxed for 1 hour at 100° C. After cooling to room temperature, the supernatant was separated from undigested solids by centrifugation and diluted to 200 mL with deionized water. The Center for Applied Isotope Studies (CAIS) at UGA determined the elemental composition of the sediment extracts using a VG PlasmaQuad 3 inductively coupled plasma mass spectrometer (Thermo Scientific, Waltham, Mass.).

Enrichment Culture. The basal salts medium (BSM) used for enrichment cultures contained the following ingredients (in grams per liter of deionized H₂O): K₂CO₃×1.5H₂O, 0.240; KH₂PO₄, 0.100; K₂HPO₄, 0.150; NH₄Cl, 0.075; vitamin solution (Oremland et al., 1994, Appl. Environ. Microbiol., 60:3011-3019), 10 mL; and SL-10 trace metals solution (Widdel et al., 1983, Arch. Microbiol., 134:286-294) supplemented with 14 mM MgCl₂ and 700 μM CaCl₂, 1 mL. The pH of the medium was adjusted to 7.0 with HCl and it was made anaerobic by incubating in a Coy anaerobic chamber (Coy Laboratory Products, Grass Lake, Mich.) containing an atmosphere of 95% N₂/5% H₂ for at least 96 hours prior to use. Sterile stock solutions of ACS reagent-grade sodium L-lactate, KSb(OH)₆, and Na₂S×9H₂O were prepared in deionized water and made anaerobic in the same fashion as described above. All subsequent manipulations were performed within the anaerobic chamber.

Inocula for enrichment cultures were taken from a depth of 5-10 cm within the sediment core. The enrichments were initiated by adding ˜1.0 g (wet weight) of sediment to a total of 100 mL BSM amended with 2 mM KSb(OH)₆, 1 mM sodium L-lactate, and 100 μM Na₂S×9H₂O (as reducing agent and sulfur source) in sterile 160-mL glass serum bottles. The serum bottles were then closed with sterilized butyl rubber stoppers and crimp-sealed with aluminum caps before being placed in the dark at 30° C. without shaking. Every week, or after the medium had become noticeably turbid to the naked eye, enrichments were sub-cultured by dispensing 10 mL of culture into 90 mL of fresh BSM and incubating as described above. A pure culture of a candidate antimonate-reducing organism was obtained by three successive rounds of dilution streaking onto anaerobic BSM agar plates (1.5% w/v) supplemented with 2 mM antimonate, 1 mM lactate, and 100 μM Na₂S×9H₂O.

Growth Experiments. The isolate was systematically tested for the ability to use antimonate as a TEA for anaerobic respiration with lactate as the sole carbon and energy source. Cells grown to mid-log phase were collected by centrifugation under an atmosphere of 95% N₂/5% H₂ and re-suspended in fresh BSM. Aliquots of this suspension were dispensed in triplicate into each of three sets of 200 mL glass serum bottles containing BSM medium. The first set of serum bottles contained BSM amended with 2 mM antimonate alone; the second set, 1 mM lactate alone; and the third set, both 2 mM antimonate and 1 mM lactate but with a heat-killed inoculum that had been subjected to two successive rounds of autoclaving. These first three sets of triplicate incubations served as negative controls. To a fourth set of triplicate serum bottles, an aliquot of washed, live cells was added to BSM medium amended with 2 mM antimonate and 1 mM lactate. The bottles were then closed with butyl rubber stoppers and sealed with aluminum crimp caps before incubating at 30° C. in the dark without shaking. At regular intervals, an aliquot of culture was extracted using a sterile needle and syringe for analysis of cell density, antimonate/antimonite concentrations, and lactate/acetate concentrations.

Phylogenetic Analysis. Genomic DNA was extracted from a cell pellet using a phenol-chloroform method as described elsewhere (Ferrari et al., 1999, Hydrobiologia, 401:55-68). The concentration and purity of extracted DNA was measured on a NanoDrop 2000c UV-Vis spectrophotometer (Thermo Scientific). The polymerase chain reaction (PCR) was used to amplify a large region of the 16S rRNA gene using universal bacterial primers 27F and 1492R (Lane, D. J., 1991, In Nucleic Acid Techniques in Bacterial Systematics; Stackebrandt, E., Goodfellow, M., Eds.; John Wiley and Sons: Hoboken, N.J., pp. 115-175). Bands corresponding to PCR products of the expected size were excised from a 1% agarose gel and purified using a QiaQuik® gel extraction kit (Qiagen, Valencia, Calif.). The amplicons were sequenced in both directions using the BigDye® terminator sequencing reaction kit (Applied Biosystems, Foster City, Calif.) and the primers given above by the Georgia Genomics Facility at UGA. The 16S rRNA gene sequence was assembled using Geneious® version 6.1 (Biomatters Ltd., Auckland, New Zealand), resulting in a consensus sequence of 1,446 bp with ˜250 bp of overlap. The consensus sequence was compared to others in the GenBank database using the BLASTn algorithm (Altschul et al., 1990, J. Mol. Biol., 215:403-410) and subsequently aligned with related sequences using ClustalW (Thompson et al., 1994, Nuc. Acids Res., 22:4673-4680). A phylogenetic tree based on 1,440 bp of the 16S rRNA gene sequence was generated with bootstrap values (1,000 replicates) using the neighbor joining method (Saitou et al., 1987, Mol. Biol. Evol., 4:406-425) and Jukes-Cantor evolutionary distances (Jukes et al., 1969, T. H.; Cantor, C. R. In Mammalian Protein Metabolism; Munro, H. N., Ed.; Academic Press: Waltham, Mass., pp. 21-132). The partial 16S rRNA gene sequence obtained in this study has been deposited in NCBI Genbank under accession number KF387535.

Microscopy. Light and epifluorescence microscopy were performed using a Leica DM RXA microscope (Leica, Wetzlar, Germany). The Gram stain reaction was performed according to standard methods. Changes in cell density during growth experiments were measured by epifluorescence microscopy after staining with 0.01% (w/v) acridine orange. For scanning electron microscopy (SEM), cells were harvested by filtration onto a 0.22 μm polycarbonate membrane filter, successively fixed with 2.5% glutaraldehyde and 1% osmium tetroxide, and visualized on a Zeiss 1450EP scanning electron microscope (Carl Zeiss NTS, Peabody, Mass.) at the Center for Advanced Ultrastructural Research (CAUR) at UGA. Sb₂O₃ microcrystals were separated from spent culture medium by centrifugation at 1,000×g for 1 minute and then rinsed successively with distilled water, 100% acetone, and distilled water. The supernatant was removed after each rinse by centrifugation at 2,000×g for 2 minutes. This series was repeated for a total of three cycles. The microcrystals were then dried overnight in an oven at 65° C.

EDS, XRD, and UV-Vis/Photoluminescence Spectroscopy. Sb₂O₃ microcrystals were harvested and washed using the method described above. We assumed that the microcrystals were substantially free of cells and cellular debris after the final washing step, as cells were not evident when the preparations were examined using SEM. Elemental analysis of Sb₂O₃ microcrystals by energy dispersive x-ray spectroscopy (EDS) was performed using an Oxford Instruments X-Act 10 mm² Silicon Drift Detector (Oxford Instruments, Concord, Mass.) attached to the Zeiss 1450EP SEM at the CAUR. The microcrystal samples were spread onto a conductive carbon tab and a thin carbon fiber coating was applied in preparation for EDS. X-ray powder diffraction (XRD) analysis was performed at the X-ray Diffraction Lab at UGA using a Bruker D8 Advance X-ray powder diffractometer (Bruker AXS, Karlsruhe, Germany) with CoKa radiation (λ=1.789 Å) operating at 40 kV and 40 mA. The room temperature UV-visible absorption spectrum of a suspension of Sb₂O₃ microcrystals in ethanol was recorded on an Evolution 201 UV-Vis spectrophotometer (Thermo Scientific) equipped with a xenon flash lamp. The room temperature photoluminescence spectrum was measured with an Aqualog spectrofluorometer (Horiba Scientific, Edison, N.J.) at an excitation wavelength of 325 nm.

Chemical Analyses. Lactate and acetate concentrations were measured by high performance liquid chromatography (HPLC) using a mobile phase of 0.016 N sulfuric acid and a UV detector set at a wavelength of 210 nm (Culbertson et al., 1988, Orig. Life Evol. Biosph., 18:397-407). Antimonite and antimonate concentrations were determined after selective removal of antimonite by a liquid-liquid extraction technique (Han-wen et al., 1982, Talanta, 29:589-593, Garboś, 2000, Spectrochim. Acta Part B: Atom. Spectrosc., 55:795-802). Briefly, a 2 mL aliquot of culture was dispensed into a 5 mL centrifuge tube and acidified with 0.5 mL of 4.5 M HCl. Following acidification, 0.5 mL of a 100 mM solution of N-benzoyl-N-phenylhydroxylamine (BPHA) in chloroform was added to the tube and the mixture was vortexed for 15 minutes at room temperature. Between pH 0.3-6, antimonite forms a non-ionic complex with poorly water-soluble BPHA, quantitatively extracting antimonite into the organic phase, whereas no extraction of antimonate occurs within this range (Chen et al., 1983, J. Chinese Chem. Soc., 30:249-253, Lyle et al., 1966, Anal. Chim. Acta., 36:286-297). Following extraction, the concentration of Sb remaining in the aqueous phase (corresponding to antimonate) was measured by the CAIS at UGA using a Thermo Jarrell-Ash 965 inductively coupled argon plasma optical emission spectrometer (Thermo Jarrell-Ash, Franklin, Mass.) at an emission wavelength of 206.83 nm. The antimonite concentration was then determined by subtracting the measured antimonate concentration from the total amount of antimonate initially added to the culture medium. This subtractive method was used because direct measurement of antimonite proved extremely difficult due to precipitation of Sb₂O₃ along the inner surfaces of serum bottles.

### Results and Discussion:

Sediment Analysis. The total metal content of the NBWS-1 subsurface sediments is presented in Table 2. The sediments are enriched in several metal(loid)s, including As, Mo, Se, Te, and Sb relative to average concentrations obtained for the upper continental crust (Hans Wedepohl, K. 1995, Geochim. Cosmochim. Acta., 59:1217-1232, Hu et al., 2008, Chem. Geol., 253:205-221). It is important to note, however, that the Sb concentration measured in these sediments falls within the background range of 0.06-8.8 ppm observed for a variety of unpolluted soils from the United States, Europe, and Australia (Wilson et al., 2010, Environ. Pollut., 158:1169-1181). Sb concentrations from contaminated sites range from 7.4-15,100 ppm for soils impacted by current or past mining activities to 35-17,500 for shooting range soils (Wilson et al., 2010, Environ. Pollut., 158:1169-1181).

TABLE 1
Concentrations of selected metal(loid)s in subsurface
(5-10 cm depth) sediments receiving hydrological
inputs from NBWS-1 and comparison to mean
abundance in the upper continental crust.

Concentration (ppm)

| Element | NBWS-1 Sediments^{a} | Upper Continental Crust^{b} |
|---|---|---|
| As | 38.91 ± 0.52 | 2.00 |
| Zn | 9.71 ± 0.95 | 52.0 |
| Cu | 7.24 ± 4.49 | 14.3 |
| Ni | 2.58 ± 0.12 | 18.6 |
| Mo | 1.86 ± 0.12 | 1.40 |
| Cr | 1.36 ± 0.03 | 35.0 |
| Pb | 0.85 ± 0.11 | 17.0 |
| Sb | 0.77 ± 0.05 | 0.31 |
| Co | 0.67 ± 0.09 | 11.6 |
| Se | 0.24 ± 0.05 | 0.083 |
| Te | 0.09 ± 0.03 | 0.027^{‡} |
| Cd | 0.09 ± 0.01 | 0.102 |

^{a}Average ± SD for three replicate samples
^{b}Data from Wedepohl (1995)
^{‡}Value taken from Hu and Gao (2008)

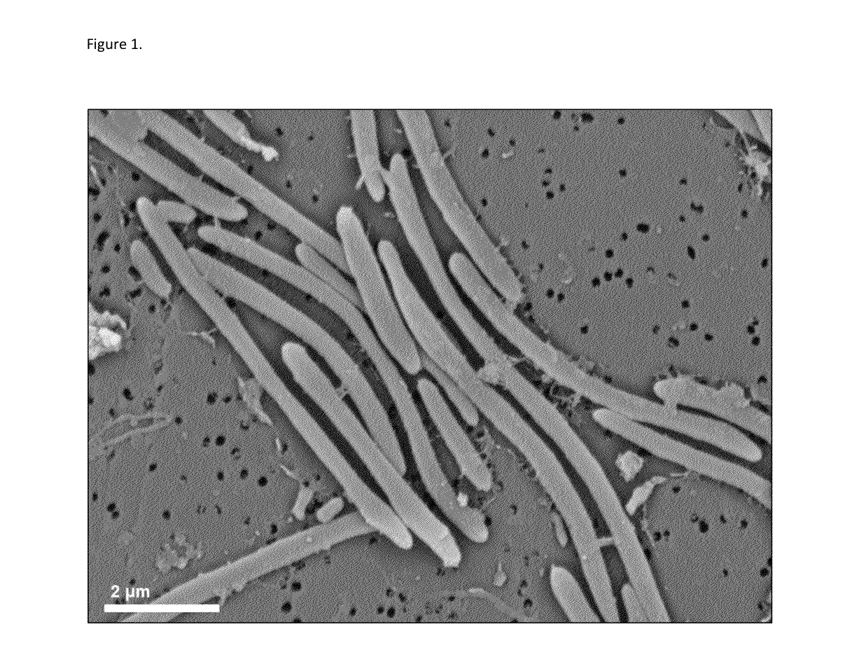

Enrichment Culture and Isolation of an Sb(V)-Respiring Bacterium. Incubation of NBWS-1 sediments under anoxic conditions with millimolar concentrations of lactate and antimonate as the electron donor and acceptor pair, respectively, resulted in the stimulation of microbial growth relative to unamended controls. The enrichment culture was transferred repeatedly over a period of six months until it was observed to be dominated by a single motile, endospore-forming, and rod-shaped morphotype. This organism stained Gram negative when subjected to Gram staining, but its phylogeny indicated a Gram positive cell envelope architecture was expected. Indeed, when the cells were observed using transmission electron microscopy, the architecture typical of a Gram positive microbe was observed (see Example 2). The organism, which formed small, off-white colonies on agar media, was isolated into pure culture and designated strain MLFW-2. SEM visualization of a culture of strain MLFW-2 grown on antimonate and lactate showed that cells form filaments of highly variable length during growth (FIG. 1).

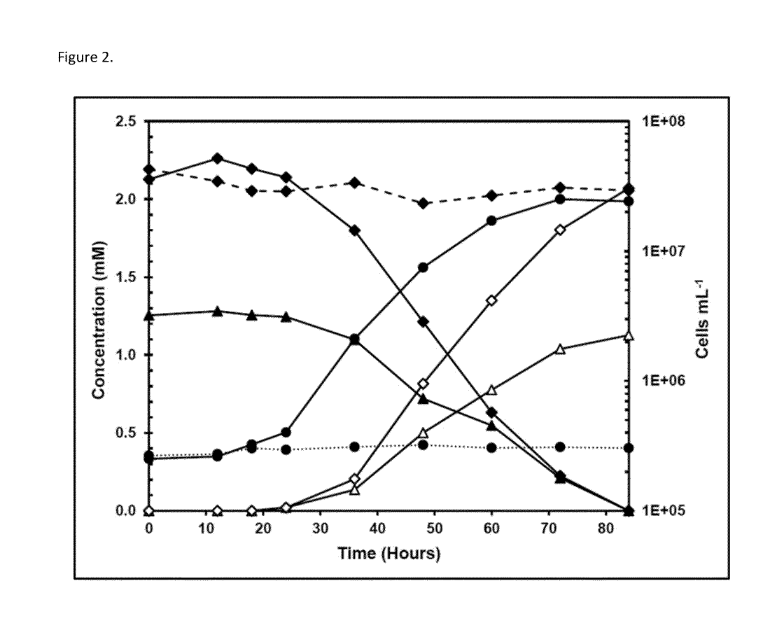

Growth Experiments. Strain MLFW-2 was capable of completely respiring a 2 mM amendment of antimonate in approximately 80 hours at 30° C. using lactate as the sole carbon and energy source (FIG. 2). Antimonate reduction was accompanied by an equivalent rise in the concentration of antimonite, while lactate was stoichiometrically oxidized to acetate and presumably HCO₃⁻ according to the reaction: Lactate⁻+2Sb(OH)₆⁻+H⁺→Acetate⁻+HCO₃⁻+2Sb(OH)₃⁰+4H₂O (ΔG′=−199.7 kJ mol⁻¹). These activities coincided with a roughly 100-fold increase in cell density, indicating that strain MLFW-2 conserved energy from this process. This reaction was biological in nature, as antimonate reduction was not observed in controls lacking lactate or in which cells were heat-killed prior to inoculation, and no growth was obtained on lactate alone (FIG. 2).

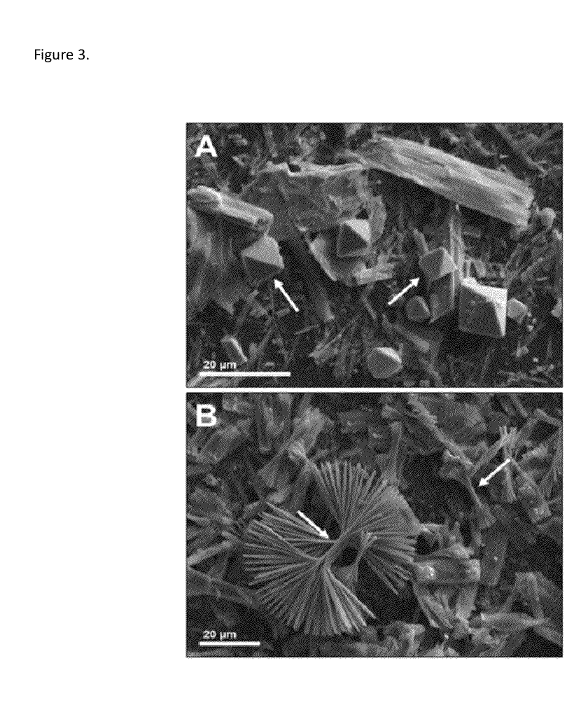

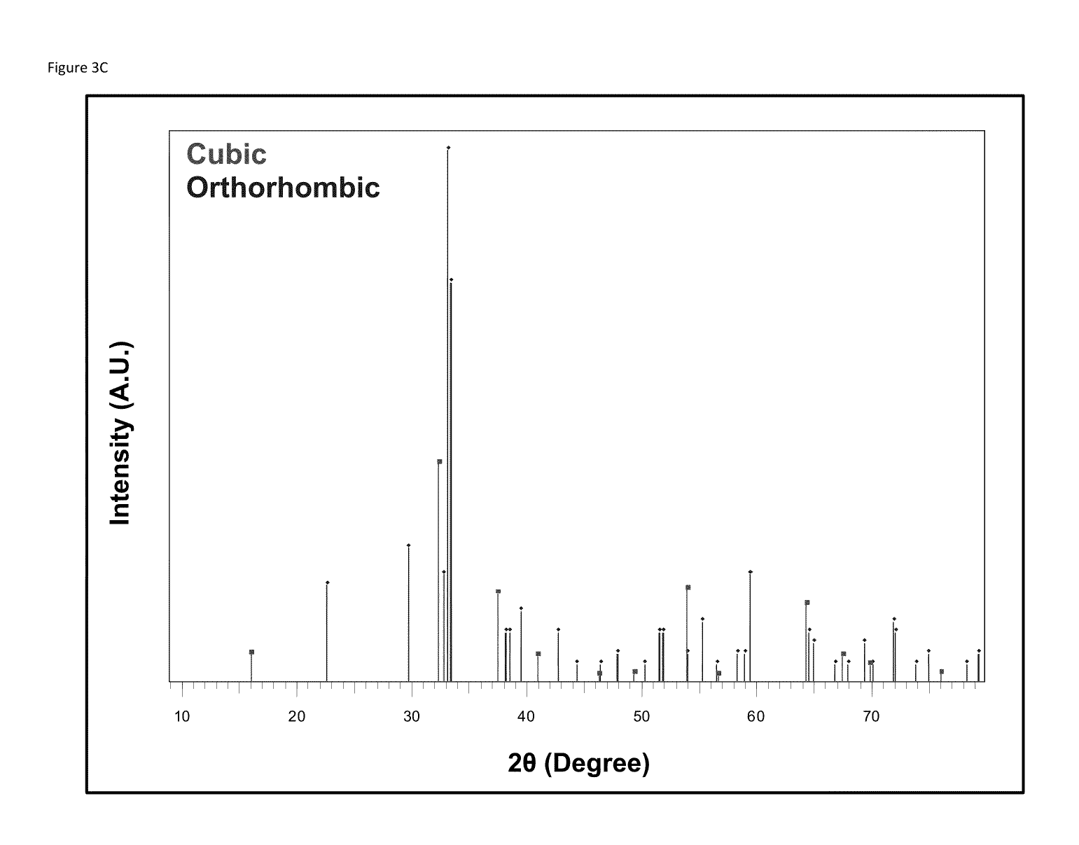

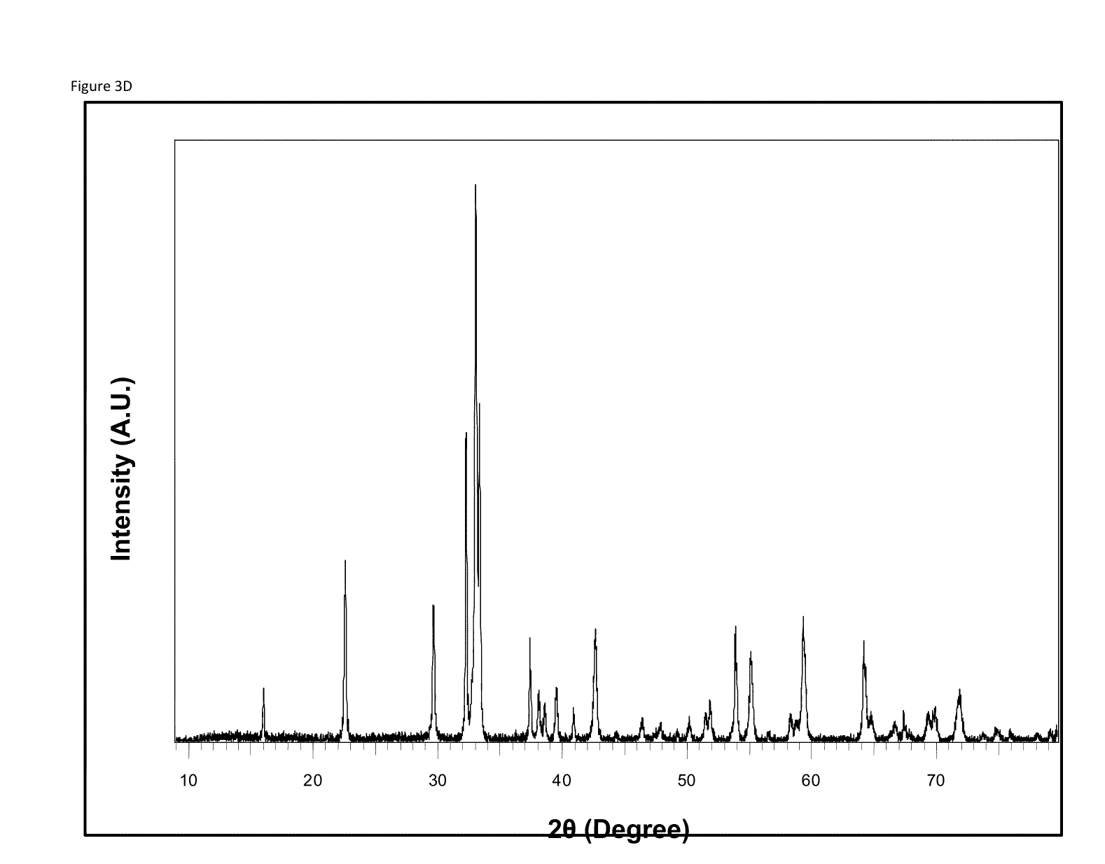

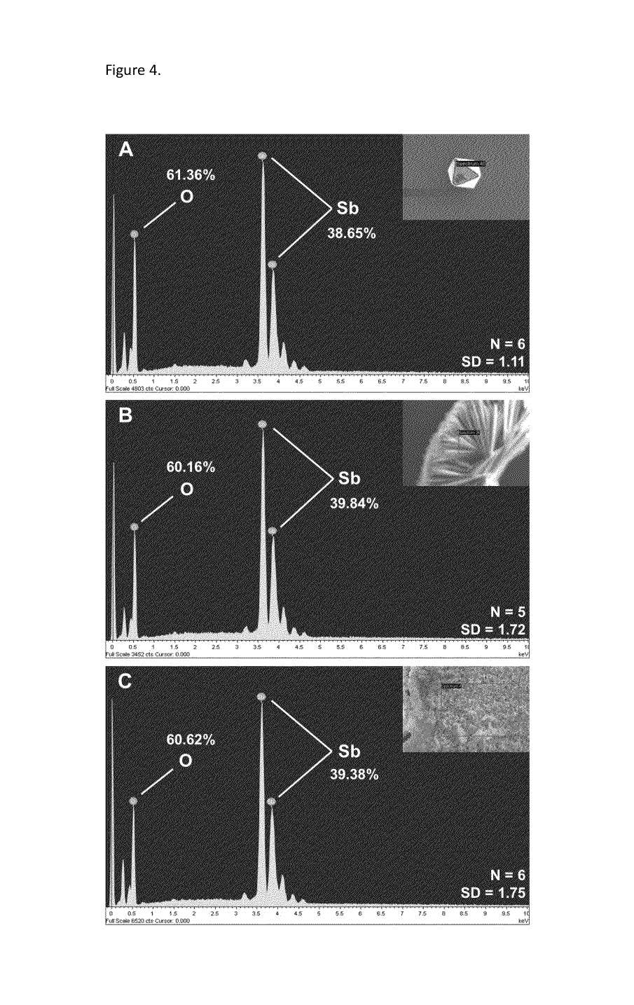

Antimonate reduction was followed by the precipitation and accumulation of a white, crystalline substance. Visualization of the precipitate by SEM showed that it consisted of an unequal mixture of two crystal phases—a less common cubic phase and a more prevalent prismatic phase displaying complex “bowtie” morphology. The cubic crystals were quite uniform and ranged in size from 5-20 μm (FIG. 3A). The “bowtie”-shaped crystals were generally 10-60 μm long and composed of blade-like projections radiating outward from each end of a central bundle (FIG. 3B). Elemental analysis of both crystal phases by energy-dispersive X-ray spectroscopy (EDS) showed that they were comprised of Sb and O in atomic ratios consistent with antimony trioxide (Sb₂O₃) (FIGS. 4A and B). The EDS spectrum of commercially-available, ≧99% pure Sb₂O₃ was identical to the spectrum of the precipitate from our cultures (FIG. 4C). Sb₂O₃ naturally occurs in two polymorphs, cubic (sénarmontite) and orthorhombic (valentinite). Sénarmontite is the most stable polymorph at temperatures up to 570° C., while valentinite was thought to be stable only at higher temperatures (Biver et al., 2013, Geochim. Cosmochim. Acta, 109:268-279). However, valentinite is actually metastable and can be prepared in the laboratory at room temperature. X-ray diffraction (XRD) confirmed that the precipitate from our cultures consisted of a mixture of cubic and orthorhombic Sb₂O₃ (FIGS. 3C and D). In light of these observations, the immediate product of antimonate reduction is likely the amphoteric hydroxide Sb(OH)₃⁰, which subsequently loses water and precipitates as sénarmontite and valentinite according to the reaction: Sb(OH)₃⁰Sb₂O₃₍ₛ₎+3H₂O (Zotov et al., 2003, Geochim. Cosmochim. Acta., 67:1821-1836).

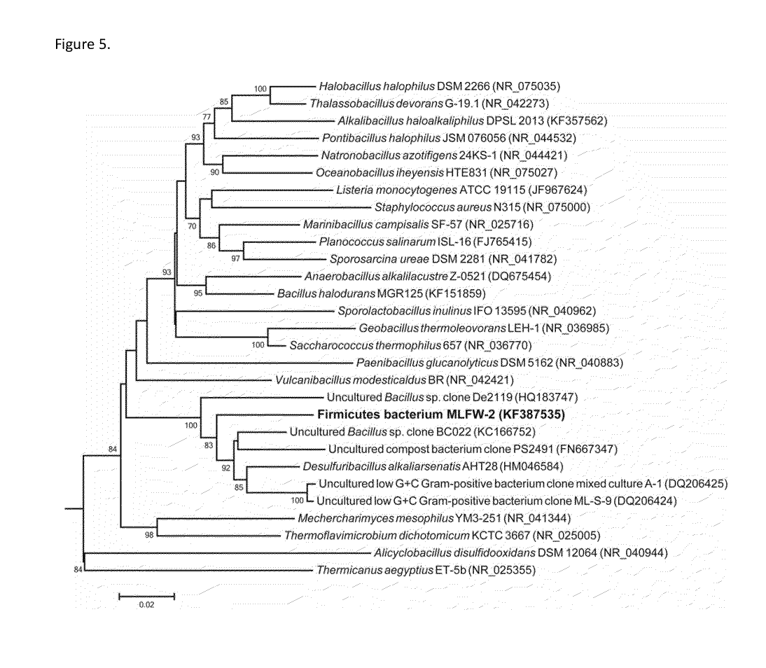

Phylogenetic Analysis. Phylogenetic analysis of a 1,450 bp region of the 16S rRNA gene of strain MLFW-2 revealed the isolate to be a member of a novel, deeply branching family within the order Bacillales of the phylum Firmicutes (FIG. 5). The MLFW-2 16S rRNA gene shares only 94% nucleotide identity with that of its closest described relative, Desulfuribacillus alkaliarsenatis AHT28, an obligately anaerobic, dissimilatory sulfur- and arsenate-reducing haloalkaliphile isolated from a Russian soda lake (Sorokin et al., 2012, Extremophiles, 16:597-605). As with strain MLFW-2, this organism exhibits a curved, rod-shaped morphology and forms round, terminal endospores. The 16S rRNA gene of strain MLFW-2 also shares 93% sequence identity with two low G+C Gram-positive sequences, “clone ML-S-9” and “clone mixed culture A-1”, retrieved from enrichments of Mono Lake water samples amended with arsenate and sulfide (Hollibaugh et al., 2006, Appl. Environ. Microbiol., 72:2043-2049).

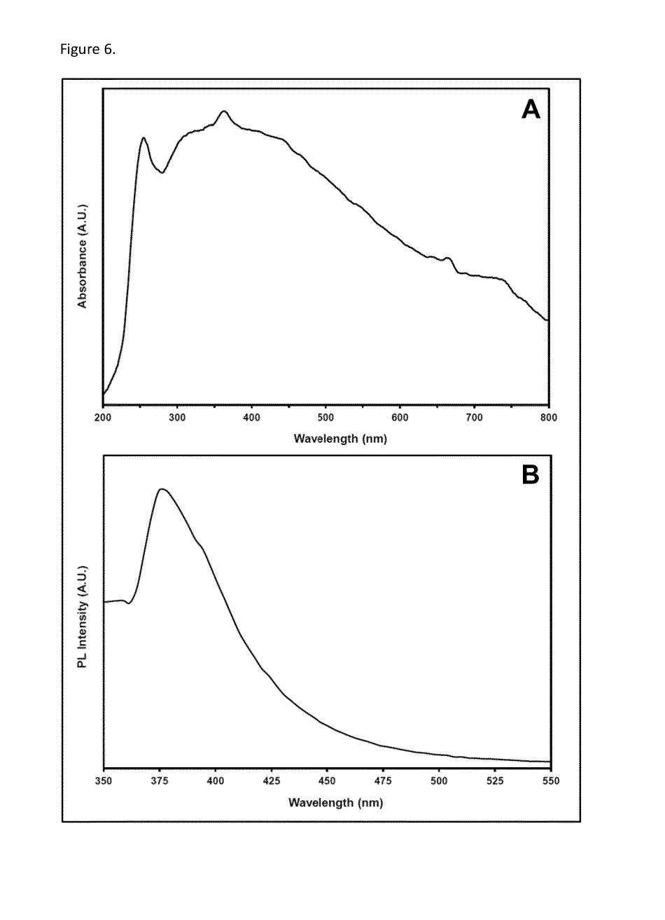

Optical Properties of Sb₂O₃ Microcrystals. We analyzed the optical properties of Sb₂O₃ precipitated in cultures of strain MLFW-2 to compare them with those of crystals prepared by chemical syntheses. The room temperature UV-Vis absorption spectrum measured from 200-800 nm showed that the biologically-produced Sb₂O₃ provide a broad absorption within this range with two prominent maxima at 255 nm and 364 nm (FIG. 6A). These values are in close proximity to those reported in the literature for artificially-synthesized Sb₂O₃ “nano-rods” and “nano-belts,” as well as “bowtie”- and “flower”-shaped microstructures (Ge et al., 2010, J. Alloys Comp., 494:169-174, Deng et al., 2006, J. Phys. Chem. B, 110:18225-18230, Li et al., 2013, Mater. Res. Bull., 48:1281-1287, Ge et al., 2013, J. Solid State Chem., 200:136-142). The peak at 255 nm corresponds to a λₒₙₛₑₜ of 372.6 nm and a calculated optical band gap (E_{g}) of 3.33 of eV, which is very similar to that of bulk orthorhombic phase Sb₂O₃ (λₒₙₛₑₜ=375 nm; E_{g}=3.30 eV) (Geng et al., 2011, Phys. Status Solidi C., 8:1708-1711). Moreover, the room temperature photoluminescence spectrum recorded at an excitation wavelength of 325 nm showed that the Sb₂O₃ microcrystals exhibit a strong photoluminescence maximum at ˜378 nm (FIG. 6B). Once again, this value is in agreement with others reported in the literature for Sb₂O₃ nano- and micro-structures of diverse morphologies synthesized for use in the semi-conductor industry (Ge et al., 2010, J. Alloys Comp., 494:169-174, Deng et al., 2006, J. Phys. Chem. B, 110:18225-18230, Li et al., 2013, Mater. Res. Bull., 48:1281-1287).

Taken together, our data demonstrate that the Sb₂O₃ microcrystals produced in cultures of strain MLFW-2 possess properties that may be of commercial significance to the nanotechnology, optics, and electronics industries. As an important member of the Group V-VI semiconductors, Sb₂O₃ has been proposed as a promising material for optical applications due to its wide optical band gap, low melting point, low phonon energy, and high refractive index (Cebriano et al., 2012, Mater. Chem. Phys., 135:1096-1103). In recent years, a series of Sb₂O₃ crystals with novel morphologies have been synthesized using various strategies, of which the solvothermal and hydrothermal routes remain the most widely used. However, these processes can often be slow, expensive, or require elevated temperatures or the use of noxious reagents, thus leading to the creation of toxic waste streams (Jha et al., 2009, Biochem. Eng. J., 43:303-306). We have now shown that commercially significant Sb₂O₃ microstructures can be synthesized using a potentially inexpensive, relatively non-toxic, and sustainable method. Thus, DSbR has the potential to serve as an alternative to chemical routes for the controlled synthesis of such microstructures.

In conclusion, the results presented here demonstrate for the first time that microorganisms are capable of anaerobic respiration using antimonate as a TEA. This discovery completes the biogeochemical cycle of Sb through the oxidized and reduced forms and adds Sb to the growing list of metals and metalloids whose oxidized species are capable of serving as electron acceptors for microbial respiration—a list that already includes Fe, Mn, Cr, U, V, Co, Tc, Np, Pu, As, Se, and Te. Importantly, the insoluble product of antimonate reduction, Sb₂O₃, is a versatile compound that may be of commercial significance to the emerging nanotechnology industry.

### Example 2

### Arcibacillus stibiireducens gen. nov., sp. nov., an Obligately Anaerobic, Dissimilatory Antimonate-Reducing Bacterium Isolated from Anoxic Sediments

Based on phylogenetic, phenotypic, and chemotaxonomic evidence, strain MLFW-2, referred to in this Examples as MLFW-2^{T}, represents a novel species of a new genus within the order Bacillales, for which the name Arcibacillus stibiireducens gen. nov., sp. nov. is proposed. The type strain of the type species is MLFW-2^{T} (=ATCC PTA-120556^{T}).

Antimony (Sb) is a trace element widely distributed throughout the environment as a result of both natural processes and anthropogenic activities. The biogeochemistry of Sb has long been an understudied topic, even though it is toxic and its use dates back several thousand years (Sundar et al., 2010, Int J Environ Res Public Health, 7:4267-4277; Anderson, C. G., 2012, Chem Erde-Geochem., 72:3-8). Information on Sb speciation and mobility in the environment is lacking and this is of particular concern in light of recent data demonstrating an enrichment of atmospheric Sb dating back from the Roman period (Shotyk et al., 2004, Global Biogeochem Cycles, 18:GB1016; Filella et al., 2009, Environ Chem., 6:95-105). These findings, along with highly publicized studies demonstrating considerable leaching of Sb from plastic drinking water bottles and juice containers, have led some to question whether Sb has become “the new lead” (Shotyk, et al., 2006, J Environ Monit., 8:288-292; Fox, M., 2006, Is antimony the new lead? In Highlights in Chemical Science, vol. 2: Royal Society of Chemistry).

Under oxic and anoxic conditions, thermodynamic calculations predict that Sb should primarily occur as the antimonate [Sb(V)] and antimonite [Sb(III)]oxyanions, respectively (Wilson et al., 2010, Environ Pollut., 158:1169-1181). In spite of these predictions, significant concentrations of thermodynamically unstable species have been measured in the environment and biological activity has been invoked as a possible source (Filella et al., 2002, Earth-Sci Rev., 57:125-176). Indeed, a number of phylogenetically-diverse antimonite-oxidizing bacteria have already been isolated (Lyalikova, N. N., 1974b, Mikrobiol. 43:941-948; Hamamura et al., 2013, Microbes Environ., 28:257-263; Lehr et al., 2007, Appl Environ Microbiol., 73:2386-2389; Li et al., 2013, Int Biodeterior Biodegrad., 76:76-80). Of these organisms, only the chemolithoautotrophic Stibiobacter senarmontii VKM MV-1130^{T} was shown to conserve energy from the oxidation of antimonite (Lyalikova, N. N., 1974a, (1974a). Dokl Akad Nauk SSSR 205:1228-1229).

On the other hand, data regarding the involvement of microorganisms in the reductive side of the Sb biogeochemical cycle have remained elusive. We published the first account of a dissimilatory antimonate-reducing bacterium, strain MLFW-2^{T}, that was capable of respiring antimonate using lactate as the electron donor (Abin et al., 2014, Environ Sci Technol, 48:681-688; Example 1). Reduction of antimonate by strain MLFW-2^{T} was accompanied by the precipitation of antimonite as cubic and orthorhombic microcrystals of antimony trioxide (Sb₂O₃). Here we describe the morphological, physiological, and phylogenetic characteristics of strain MLFW-2^{T} and show that it represents a novel species of a new genus belonging to the order Bacillales. The superscript “T” indicates that MLFW-2 is the “type strain” of the new species Arcibacillus stibiireducens (i.e. the first strain of this species to be discovered and described, providing the defining features of the new taxon). We also reevaluate the taxonomic status of its closest cultured relative and propose to reassign it to this new genus. Strain MLFW-2^{T} was isolated from anoxic sediments receiving freshwater hydrological inputs from a warm spring near Mono Lake, Calif. Trace metals analysis of these sediments revealed significant enrichment of arsenic in addition to slightly elevated levels of antimony, selenium, and tellurium (Abin et al., 2014, Environ Sci Technol, 48:681-688). A lactate-oxidizing, antimonate-reducing enrichment culture was established under anoxic conditions in a minimal low salt medium (BSM-1; Table 3, available in IJSEM Online) as described previously (Abin et al., 2014, Environ Sci Technol, 48:681-688). After six months of transfers, isolated colonies were grown on BSM-1 agar (1.5% w/v) plates. A pure culture of strain MLFW-2^{T} was obtained after three successive rounds of dilution streaking

TABLE 3
Liquid media used for the cultivation and characterization
of strain MLFW-2^{T}

| | Amount per Liter of Medium | | | | |
|---|---|---|---|---|---|
| Component | BSM-1 | BSM-2 | BSM-3^{a} | BSM-4^{b} | BSM-5 |
| NaCl | — | Variable | Variable^{‡} | Variable^{‡} | 3.62 g |
| NaHCO₃ | — | — | — | Variable^{§} | 4.20 g |
| Na₂CO₃ | — | — | — | Variable^{§} | — |
| K₂CO₃ × | 0.240 g | 0.240 g | — | — | — |
| 1.5H₂O | | | | | |
| NaH₂PO₄ | — | — | Variable^{†} | — | — |
| Na₂HPO₄ | — | — | Variable^{†} | — | — |
| KH₂PO₄ | 0.100 g | 0.100 g | — | — | — |
| K₂HPO₄ | 0.150 g | 0.150 g | — | 0.261 g | 0.261 g |
| NH₄Cl | 0.075 g | — | 0.075 g | 0.075 g | 0.075 g |
| (NH₄)₂SO₄ | — | 0.079 g | — | — | — |
| Na₂SO₄ | — | — | 0.050 g | 0.050 g | 0.050 |
| Na₂S × | 0.024 g | 0.024 g | 0.024 g | 0.024 g | 0.024 g |
| 9H₂O | | | | | |
| Na₂HAsO₄ × | — | 1.56 g | — | — | 1.56 g |
| 7H₂O | | | | | |
| KH₂AsO₄ | — | — | 0.901 g | 0.901 g | — |
| KSb(OH)₆ | 0.526 g | — | — | — | — |
| Sodium | 0.112 g | 0.560 g | 1.12 g | 1.12 g | 1.12 g |
| L-lactate | | | | | |
| Yeast extract | — | — | 0.200 g | 0.200 g | 0.200 g |
| Vitamin | 10 mL | 10 mL | 10 mL | 10 mL | 10 mL |
| solution^{c} | | | | | |
| Trace | 1 mL | 1 mL | 1 mL | 1 mL | 1 mL |
| minerals | | | | | |
| solution^{d} | | | | | |
| Final pH | 7.0-7.5 | 7.0-7.5 | 7.0-8.0 | 8.25-10.0 | 8.2-8.5 |

^{a}Used to test pH tolerance from pH 7.0-8.0
^{b}Used to test pH tolerance from pH 8.25-10.0
^{c}Oremland et al. (1994)
^{d}Trace element solution SL-10 of Widdel et al. (1983) modified by addition of 14 mM MgCl₂ and 700 μM CaCl₂
^{‡}Final amount varied to achieve [Na⁺] = 125 mM
^{†}[H₂PO₄⁻] + [HPO₄²⁻] = 50 mM
^{§}[HCO₃⁻] + [CO₃²⁻] = 50 mM

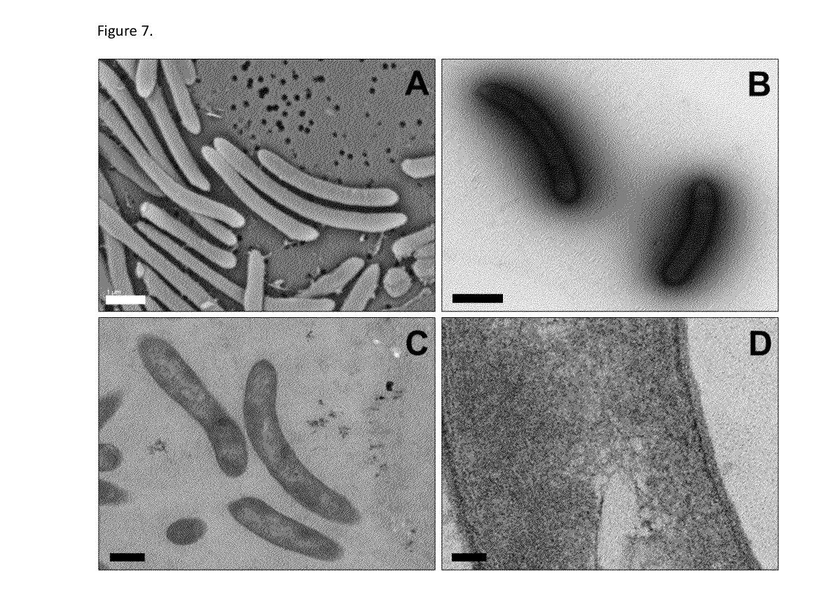

After 36-48 hours, colonies of strain MLFW-2^{T} were opaquely white and small, with diameters ranging from 0.5-0.8 mm. The colonies were circular, smooth, and convex with entire margins. Cell morphology during growth on antimonate was examined using optical light microscopy (DM RXA; Leica), scanning electron microscopy (1450EP; Zeiss), and transmission electron microscopy (Technai 20; FEI). Cells of strain MLFW-2^{T} were motile and consisted of curved rods typically 0.3-0.5 μm wide and 2.0-6.9 μm long, although cells with lengths from 7-11 μm occurred occasionally as well (FIG. 7). During the stationary phase of growth, cells formed terminal swollen sporangia containing ellipsoidal endospores. The Gram stain and KOH string test were performed on mid-exponential phase cells as previously described (Gerhardt, et al., 1994, Methods for General and Molecular Bacteriology. Washington D.C.: American Society for Microbiology; Gregersen, T., 1978, Euro J Appl Microbiol Biotechnol., 5:123-127). Strain MLFW-2^{T} stained Gram-negative and cell lysis occurred following incubation in 3% KOH. However, no apparent outer membrane was observed in ultra-thin sections and the structure of the cell envelope resembled that of Gram-positive bacteria (FIG. 7). Flagellar staining was performed according to the method of Leifson (Leifson, E., 1930, J. Bacteriol., 20:203-211). Actively growing cells of strain MLFW-2^{T} produced several peritrichous flagella.

Genomic DNA was extracted from a cell pellet using the PureLink Genomic DNA Mini Kit (Invitrogen). A fragment of the 16S rRNA gene was amplified by PCR using universal bacterial primers 27F (5′-AGAGTTTGATCCTGGCTCAG-3′ SEQ ID NO:2) and 1492R (5′-GGTTACCTTGTTACGACTT-3′ SEQ ID NO:3) (Lane, 1991, 16S/23S rRNA sequencing. In Nucleic Acid Techniques in Bacterial Systematics, pp. 115-175. Edited by E. Stackebrandt and M. Goodfellow. New York, N.Y.: John Wiley and Sons). The amplicons were sequenced directly using the BigDye Terminator v3.1 Cycle Sequencing Kit (Applied Biosystems) and an Applied Biosystems 3730×1 DNA Analyzer. The forward and reverse primers listed above were used for sequencing from both directions. The consensus 16S rRNA gene sequence of strain MLFW-2^{T} was compared with reference sequences in Genbank and the Ribosomal Database Project Release 11.1 using the BLASTn (Altschul et al. 1990, J Mol. Biol., 215:403-410) and RDP Classifier algorithms (Wang et al., 2007, Appl Environ Microbiol., 73:5261-5267), respectively. Sequence data from strain MLFW-2^{T} and its closest relatives were aligned using CLUSTALW (Thompson et al., 1994, Nuc Acids Res., 22:4673-4680). The MEGA6 software package (Tamura et al., 2013, Mol Biol Evol., 30:2725-2729) was used to generate phylogenetic trees using the neighbor-joining (Saitou et al., 1987, Mol Biol Evol., 4:406-425), maximum-parsimony (Fitch, W. M., 1971, Syst Biol., 20:406-416), and maximum-likelihood (Felsenstein, J., 1981, J Mol. Evol., 17:368-376) methods with Jukes-Cantor evolutionary distances (Jukes and Cantor, 1969, Evolution of protein molecules. In Mammalian Protein Metabolism, vol. 3, pp. 21-132. Edited by H. N. Munro. New York: Academic Press). Tree topology was evaluated by bootstrap analysis (Felsenstein, J., 1985, Evolution, 39:783-791) with 1,000 replicates.

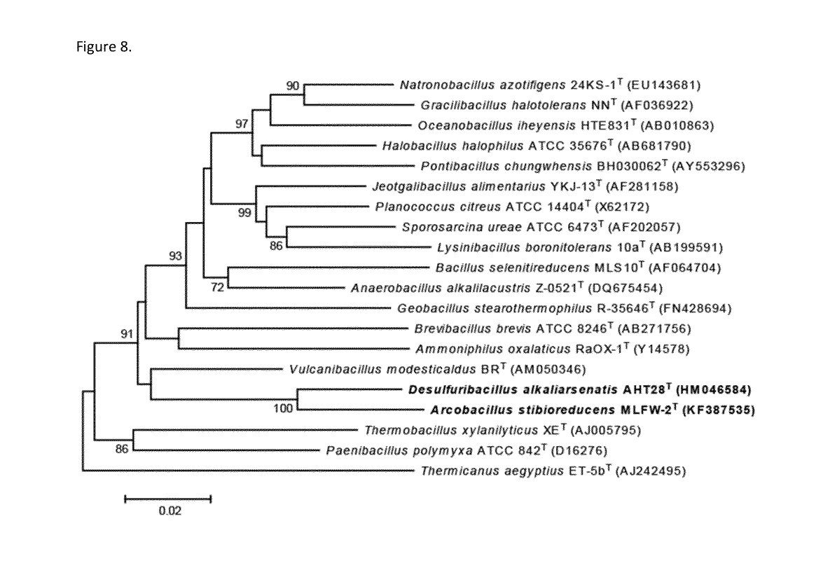

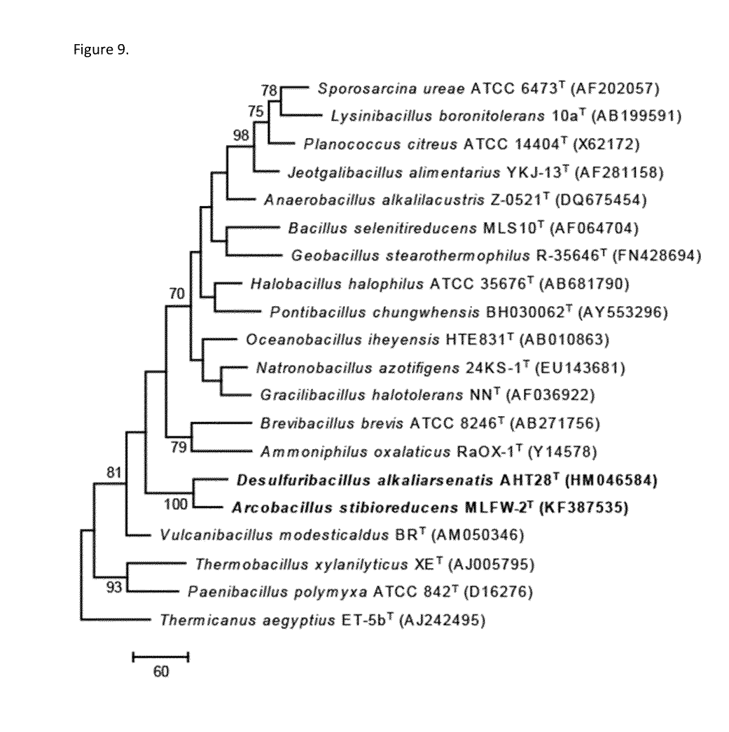

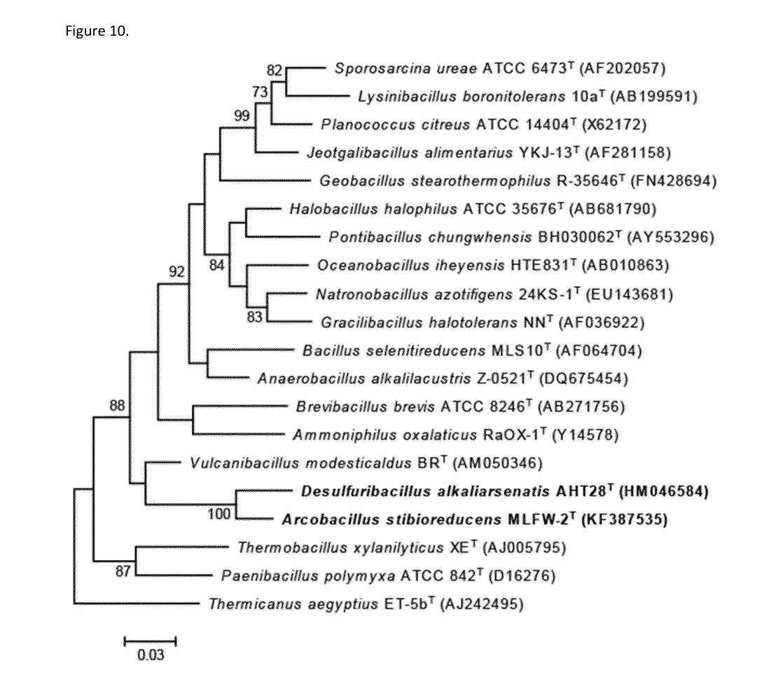

A nearly full length 16S rRNA gene sequence (1,446 nt) was obtained from strain MLFW-2^{T}. An unrooted phylogenetic tree revealed that it was a deeply-branching member of the order Bacillales, a diverse group of low G+C bacteria that stain either Gram-positive or -negative (FIG. 8). This finding was also supported by trees constructed using the maximum-parsimony and maximum-likelihood algorithms (FIGS. 9 and 10). Although the RDP Classifier query indicated a 97% probability that the sequence belonged to the Bacillales, it did not cluster with any established family. The closest phylogenetic relative of strain MLFW-2^{T} was Desulfuribacillus alkaliarsenatis AHT28, with a sequence similarity of 93.9%. D. alkaliarsenatis AHT28 is a Gram-positive, obligately anaerobic, dissimilatory sulfur- and arsenate-reducing haloalkaliphile isolated from soda lake sediments in the Kulunda Steppe, Altai, Russia (Sorokin et al., 2012, Extremophiles, 16:597-605). Strain MLFW-2^{T} consistently shared a branching node with D. alkaliarsenatis AHT28 regardless of the phylogenetic treeing method used. This relationship was supported by bootstrap values of 100% in each case (FIGS. 8, 9, and 10). Lower similarity was observed with some other type strains of the Bacillales, including Vulcanibacillus modesticaldus BR^{T} (89.7%), Paenibacillus taiwanensis BCRC 17411^{T} (88.8%), Bacillus litoralis SW-211^{T} (88.6%), Jeotgalibacillus campisalis SF-57^{T} (88.6%), and Jeotgalibacillus marinus 581^{T} (88.5%). The 16S rRNA gene of strain MLFW-2^{T} was also related to uncultured bacterial clones retrieved from an anaerobic aquifer (Genbank accession number KC166752; similarity value 94.7%), municipal compost pile (FN667347; 92.9%), alkaline, hypersaline lake (DQ206424 and DQ206425; 92.8% and 92.7%, respectively), and leachate sediment (HQ183747; 91.5%).

The DNA G+C content of strain MLFW-2^{T} was determined by whole genome sequencing. A draft genome sequence with approximately 150× coverage was generated using 2×250 bp paired-end sequencing on the Illumina MySEQ platform. Adaptor removal and quality trimming (>Q30) was performed using PRINSEQ version 0.20.4 (Schmieder et al., 2011, Bioinformatics, 27:863-864). The remaining paired-end reads were assembled into contigs de novo using Soapdenovo version 1.05 (Li et al., 2009, Genome Res., 20:265-272) with a kmer setting of 127. The DNA G+C content of strain MLFW-2^{T} was found to be 38.2 mol %. This value is similar to the 39.1 mol % reported for D. alkaliarsenatis AHT28 (Sorokin et al., 2012, Extremophiles, 16:597-605).

Growth experiments aimed to characterize the physiological properties of strain MLFW-2^{T} were all carried out in duplicate at 30° C., except where noted. Tests for the utilization of electron donors and acceptors were performed in modified BSM-1 from which lactate and antimonate were omitted from the medium. Growth under different conditions was assessed by monitoring cell density using acridine orange staining and epifluorescence microscopy (Hobbie et al., 1977, Appl Environ Microbiol., 33:1225-1228). For a positive result, growth had to reach a level of at least two-fold that of the negative control and had to maintain that increase relative to the negative control for three consecutive transfers.

An investigation into the range of terminal electron acceptors utilized by strain MLFW-2^{T} was conducted in the presence of 10 mM lactate as electron donor. The following electron acceptors were tested: nitrate (5 mM), nitrite (2 mM), amorphous Fe(III) oxyhydroxide (5 g l⁻¹), Fe(III) citrate (5 mM), colloidal MnO₂ (5 g L⁻¹), sulfate (5 mM), sulfite (2 mM), tetrathionate (5 mM), thiosulfate (5 mM), elemental sulfur (5 g l⁻¹), arsenate (5 mM), selenate (5 mM), selenite (5 mM), tellurate (1 mM), tellurite (1 mM), chromate (1 mM), vanadate (5 mM), molybdate (5 mM), fumarate (5 mM), dimethylsulfoxide (DMSO) (5 mM), and trimethylamine-N-oxide (TMAO) (5 mM). All electron acceptors were added from anoxic, filter-sterilized stock solutions. The ability to grow aerobically (21% O₂) was tested in 200 mL Erlenmeyer flasks open to the atmosphere. The test for microaerophilic growth was conducted in stoppered glass serum bottles to which sterile air was injected into the headspace at a final O₂ concentration of 2% (v/v). Amorphous Fe(III) oxyhydroxide was prepared according to the method of (Lovley et al., 1988, Appl Environ Microbiol., 54:1472-1480). Colloidal MnO₂ was synthesized according to the method of (Lovley et al., 1988, Appl Environ Microbiol., 54:1472-1480).

In addition to antimonate, strain MLFW-2^{T} was able to grow using nitrate, nitrite, arsenate, selenate, selenite, tellurate, and DMSO as terminal electron acceptors. In the presence of lactate, growth on each of these alternative electron acceptors was enhanced over that on antimonate under the same conditions. In the case of nitrate, it was sequentially reduced to nitrite and then to ammonium based on spectrophotometric analyses by the Greiss reagent (Bendschneider et al., 1951, J Marine Res., 11:87-96) and phenol-hypochlorite (Weatherburn, M. W., 1967, Anal Chem 39, 971-974) methods. Arsenate was reduced to arsenite, while selenate/tellurate was sequentially reduced to selenite/tellurite and then to elemental selenium/tellurium. The detection and quantification of arsenic, selenium, and tellurium oxyanions was accomplished using high performance liquid chromatography (Fisher et al., 2007, Environ Sci Technol., 42:81-85; Fisher et al., 2008, Appl Environ Microbiol., 74:2588-2594). No growth was observed in the presence of both 21% and 2% O₂, indicating that strain MLFW-2^{T} was an obligate anaerobe. D. alkaliarsenatis AHT28 is also a strict anaerobe capable of respiring arsenate, although elemental sulfur and thiosulfate can also support growth (Sorokin et al., 2012, Extremophiles, 16:597-605). A key difference between the two strains lies in the inability of D. alkaliarsenatis AHT28 to grow using nitrate, nitrite, selenate, selenite, or DMSO as an electron acceptor.

Growth experiments aimed to characterize the physiological properties of strain MLFW-2^{T} were all carried out in duplicate at 30° C., except where noted. Tests for the utilization of electron donors and acceptors were performed in modified BSM-1 from which lactate and antimonate were omitted from the medium. Growth under different conditions was assessed by monitoring cell density using acridine orange staining and epifluorescence microscopy (Hobbie et al., 1977, Appl Environ Microbiol., 33:1225-1228). For a positive result, growth had to reach a level of at least two-fold that of the negative control and had to maintain that increase relative to the negative control for three consecutive transfers.

An investigation into the range of terminal electron acceptors utilized by strain MLFW-2^{T} was conducted in the presence of 10 mM lactate as electron donor. The following electron acceptors were tested: nitrate (5 mM), nitrite (2 mM), amorphous Fe(III) oxyhydroxide (5 g l⁻¹), Fe(III) citrate (5 mM), colloidal MnO₂ (5 g L⁻¹), sulfate (5 mM), sulfite (2 mM), tetrathionate (5 mM), thiosulfate (5 mM), elemental sulfur (5 g l⁻¹), arsenate (5 mM), selenate (5 mM), selenite (5 mM), tellurate (1 mM), tellurite (1 mM), chromate (1 mM), vanadate (5 mM), molybdate (5 mM), fumarate (5 mM), dimethylsulfoxide (DMSO) (5 mM), and trimethylamine-N-oxide (TMAO) (5 mM). All electron acceptors were added from anoxic, filter-sterilized stock solutions. The ability to grow aerobically (21% O₂) was tested in 200 mL Erlenmeyer flasks open to the atmosphere. The test for microaerophilic growth was conducted in stoppered glass serum bottles to which sterile air was injected into the headspace at a final O₂ concentration of 2% (v/v). Amorphous Fe(III) oxyhydroxide was prepared according to the method of (Lovley et al., 1988, Appl Environ Microbiol., 54:1472-1480). Colloidal MnO₂ was synthesized according to the method of (Lovley et al., 1988, Appl Environ Microbiol., 54:1472-1480).

In addition to antimonate, strain MLFW-2^{T} was able to grow using nitrate, nitrite, arsenate, selenate, selenite, tellurate, and DMSO as terminal electron acceptors. In the presence of lactate, growth on each of these alternative electron acceptors was enhanced over that on antimonate under the same conditions. In the case of nitrate, it was sequentially reduced to nitrite and then to ammonium based on spectrophotometric analyses by the Greiss reagent (Bendschneider et al., 1951, J Marine Res., 11:87-96) and phenol-hypochlorite (Weatherburn, M. W., 1967, Anal Chem 39, 971-974) methods. Arsenate was reduced to arsenite, while selenate/tellurate was sequentially reduced to selenite/tellurite and then to elemental selenium/tellurium. The detection and quantification of arsenic, selenium, and tellurium oxyanions was accomplished using high performance liquid chromatography (Fisher et al., 2007, Environ Sci Technol., 42:81-85; Fisher et al., 2008, Appl Environ Microbiol., 74:2588-2594). No growth was observed in the presence of both 21% and 2% O₂, indicating that strain MLFW-2^{T} was an obligate anaerobe. D. alkaliarsenatis AHT28 is also a strict anaerobe capable of respiring arsenate, although elemental sulfur and thiosulfate can also support growth (Sorokin et al., 2012, Extremophiles, 16:597-605). A key difference between the two strains lies in the inability of D. alkaliarsenatis AHT28 to grow using nitrate, nitrite, selenate, selenite, or DMSO as an electron acceptor.

TABLE 4
Cellular fatty acid content of strain MLFW-2^{T} and
D. alkaliarsenatis AHT28 Data for D. alkaliarsenatis
AHT28 were taken from Sorokin et al. (2012). Values are
percentages of total fatty acids. Only fatty acids representing
≧1% are shown. Fatty acids representing >5% of the total
fatty acids are shown in bold. —, Not detected or <1%.

| Fatty acid | Strain MLFW-2^{T} | D. alkaliarsenatis AHT28 |
|---|---|---|
| C_{16:0} | 21.7 | 24.6 |
| C_{16:0} ALDE | — | 2.8 |
| C_{16:1}ω9 | 14.1* | 6.6 |
| C_{16:1}ω9 ALDE | — | 2.3 |
| C_{16:1}ω7 ALDE | — | 1.3 |
| C_{16:1}ω7c | 13.9 | 20.0 |
| C_{16:1}ω5 | 1.3* | 3.5 |
| iso-C_{17:0} | — | 1.1 |
| C_{18:0} | 1.2 | 1.4 |
| C_{18:1}ω9 | 7.3* | 6.0 |
| C_{18:1}ω9 ALDE | — | 1.0 |
| C_{18:1}ω7 ALDE | — | 4.1 |
| C_{18:1}ω7c | 35.4 | 20.7 |
| C_{18:1}ω5 | 1.5* | 1.4 |

*Only cis stereoisomer detected

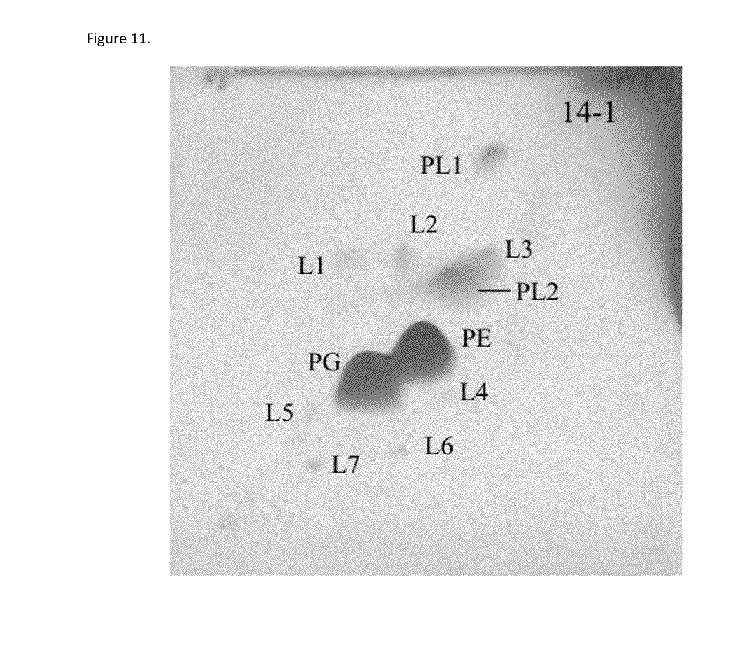

The analysis of polar lipids and respiratory lipoquinones was conducted by the Identification Service of the Deutsche Sammlung von Mikroorganismen and Zellkulturen (DSMZ, Braunschweig, Germany). The polar lipid profile of strain MLFW-2^{T} was dominated by phosphatidylglycerol and phosphatidylethanolamine (FIG. 11). Also present were moderate amounts of two unknown phospholipids (PL1 and PL2) and two unknown polar lipids (L2 and L3), as well as minor amounts of other unknown polar lipids (L1, L4-L7) (FIG. 11). The only respiratory lipoquinone that was detected was menaquinone-7 (MK-7). MK-7 is also the dominant respiratory quinone present in D. alkaliarsenatis AHT28.

The sensitivity of strain MLFW-2^{T} to various antibiotics was assessed using the same conditions employed for the chemotaxonomic analyses. Exponential phase cells were washed and then inoculated into a series of tubes containing BSM-5 supplemented with ampicillin, kanamycin, rifampicin, chloramphenicol, erythromycin, gentamicin, streptomycin, or nalidixic acid at concentrations of 5, 10, 25, 50, 75, and 100 μg/mL. The tubes were prepared in triplicate and scored for positive growth after an incubation period of 96 hours. Strain MLFW-2^{T} was only able to grow in the presence of 5 μg/mL chloramphenicol, 5-25 μg/mL kanamycin, and 5-75 μg/mL nalidixic acid.

On the basis of the morphological, physiological, phylogenetic, and chemotaxonomic characteristics presented here, we propose the creation of a novel genus, Arcobacillus gen. nov., with Arcobacillus stibioreducens sp. nov. as the type species. D. alkaliarsenatis AHT28 consistently clustered with this type species in phylogenetic analyses. While the two strains share many phenotypic traits in common, it is possible to distinguish between them using the characteristics shown in Table 5.

TABLE 5
Phenotypic comparison between strain MLFW-2^{T} and its
closest phylogenetic relative, D. alkaliarsenatis AHT28
Data for D. alkaliarsenatis AHT28 were derived from Sorokin
et al. (2012). +, Positive; −, negative; ND, not determined.
Both strain MLFW-2^{T} and D. alkaliarsenatis AHT28 are
positive for: curved, rod-shaped morphology; endospore
formation within terminal, swollen sporangia; motility by
means of several peritrichous flagella; menaquinone-7 as the
dominant respiratory quinone; and use of H₂, formate,
lactate, and pyruvate as electron donors. Both strains are
negative for: cytochrome oxidase; use of acetate, propionate,
butyrate, malate, succinate, ethanol, glucose, or fructose as
an electron donor; and use of oxygen, sulfate, sulfite,
fumarate, amorphous Fe(III) oxyhydroxide, or colloidal MnO₂
as an electron acceptor.

| | | D. alkaliarsenatis |
|---|---|---|
| Characteristic | Strain MLFW-2^{T} | AHT28 |
| Isolation source | Anoxic, | Soda lake sediment |
| | freshwater | |
| | sediment | |
| Cell width (μm) | 0.3-0.5 | 0.4 |
| Cell length (μm) | 2.0-6.9 | 2.0-7.0 |
| Gram stain | − | + |
| Endospore shape | Ellipsoidal | Round or ellipsoidal |
| Catalase | + | − |
| Temperature (° C.) | | |
| Range | 10-43 | ND-43 |
| Optimum | 34 | 35 |
| pH | | |
| Range | 7.0-10.0 | 8.5-10.6 |
| Optimum | 8.25-8.50 | 10.2 |
| NaCl concentration (%) | | |
| Range | 0-5.0 | 1.2-14.6 |
| Optimum | 0.75 | 3.5-4.7 |
| Electron donors | | |
| Lactate | + | +* |
| Pyruvate | + | +* |
| Fumarate | + | − |
| Yeast extract | + | − |
| Electron acceptors | | |
| Nitrate | + | − |
| Nitrite | + | − |
| Thiosulfate | − | +^{†} |
| Elemental sulfur | − | +^{†} |
| Arsenate | + | +^{§} |
| Antimonate | + | ND |
| Selenate | + | − |
| Selenite | + | − |
| Tellurate | + | ND |
| Tellurite | − | ND |
| DMSO | + | − |
| Major polar lipids | PG, PE^{‡} | ND |
| DNA G + C content (mol %) | 38.2 | 39.1 |

*Growth only occurs with arsenate as the electron acceptor
^{†}Growth only occurs with H₂ or formate as the electron donors
^{§}Growth only occurs with lactate or pyruvate as the electron donors
^{‡}PG, phosphatidylglycerol; PE, phosphatidylethanolamine

### Description of Arcibacillus gen. nov.

Arcibacillus (Ar'ci.ba.cil'lus. L. n. arcus arc or bow; L. masc. n. bacillus a small rod; N.L. masc. n. Arcibacillus a bow-shaped rod).

Cells occur as curved rods approximately 0.3-0.5×2.0-7.0 μm that produce terminal endospores within swollen sporangia. No outer membrane is present. Motile by means of several peritrichous flagella. Oxidase-negative. Mesophilic, with a maximum growth temperature of 43° C. Obligately anaerobic. Respiratory metabolism, using arsenate as a terminal electron acceptor. Compounds such as H₂, formate, lactate, and pyruvate are used as electron donors. No fermentative growth is observed. The major cellular fatty acids are C_{16:0}, C_{16:1}ω9, C_{16:1}ω7c, C_{18:1} ω9, and C_{18:1}ω7c. The major respiratory lipoquinone is MK-7. The G+C content of the genomic DNA ranges from 38.2 to 39.1 mol %. Strains have been isolated from anoxic sediments overlain by the effluent of a geothermal spring. 16S rRNA gene sequence analysis places Arcibacillus in the order Bacillales. The type species of the genus is Arcibacillus stibiireducens.

Description of Arcibacillus stibiireducens sp. nov.

Arcibacillus stibiireducens (sti.bi.i.re.du'cens. L. n. stibium antimony; L. part. adj. reducens converting to a different state; N.L. part. adj. stibiireducens, reducing antimony, referring to the ability to couple growth to the reduction of antimonate to antimonite).

In addition to having the characteristics given in the genus description, cells stain Gram negative and lyse upon treatment with 3% KOH. Growth is observed from 10-43° C. (optimum 34° C.), pH 7.0-10.0 (optimum 8.2-8.5), and with 0-5% (w/v) NaCl (optimum 0.75%). Cells are catalase-positive. Anaerobic respiration occurs using nitrate, nitrite, DMSO, antimonate, selenate, selenite, or tellurate as terminal electron acceptors. Sulfate, sulfite, tetrathionate, thiosulfate, elemental sulfur, chromate, vanadate, amorphous Fe(III) oxyhydroxide, Fe(III) citrate, colloidal MnO₂, molybdate, tellurite, fumarate, and TMAO cannot be utilized as electron acceptors. Formate, lactate, pyruvate, fumarate, casamino acids, yeast extract, and H₂ can serve as electron donors. Acetate, malate, succinate, maleate, oxalate, ascorbate, citrate, tartrate, glycolate, propionate, D-glucose, D-galactose, D-fructose, ethanol, methanol, glycerol, D-sorbitol, L-glycine, L-glutamate, and L-serine cannot support growth. Phosphatidylglycerol and phosphatidylethanolamine are the major polar lipids in the membrane fraction. Cells are resistant to 5 μg/mL chloramphenicol, 5-25 μg/mL kanamycin, and 5-75 μg/mL nalidixic acid, but highly sensitive to ampicillin, rifampicin, erythromycin, gentamicin, and streptomycin.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A biologically pure culture of a microbe that comprises the characteristic of reducing Sb(V) to Sb(III).

2. The biologically pure culture of the microbe of claim 1 wherein the microbe produces Sb₂O₃ by reduction of the Sb(OH)₆⁻.

3. The biologically pure culture of the microbe of claim 2 wherein the Sb₂O₃ is sénarmontite, valentinite, or a combination thereof.

4. The biologically pure culture of the microbe of claim 1 wherein the microbe further comprises the activity of reducing a compound selected from selenite, selenate, tellurate, nitrite, nitrate, arsenate, DMSO, and a combination thereof.

5. The biologically pure culture of the microbe of claim 1 wherein the microbe will not reduce a compound selected from sulfate, sulfite, thiosulfate, tetrathionate, elemental sulfur, chromate, metavanadate, iron (III) hydroxide (insoluble), iron (III)-EDTA (soluble), manganese dioxide, molybdate, chlorate, perchlorate, and a combination thereof.

6. The biologically pure culture of the microbe of claim 1 wherein the microbe comprises a 16S rRNA coding region having at least 97% identity to SEQ ID NO:1.

7. The biologically pure culture of the microbe of claim 1 wherein the microbe is a member of the order Bacillales.

8. The biologically pure culture of the microbe of claim 1 wherein the microbe has the characteristics of MLFW-2 as deposited with the American Type Culture Collection under number PTA-120556 in accordance with the provisions of the Budapest Treaty, wherein the characteristics comprise reducing Sb(v), selenite, selenate, tellurate, nitrite, nitrate, and arsenate.

9. The biologically pure culture of the microbe of claim 8 wherein the microbe is MLFW-2.

10. A composition comprising the microbe of claim 1 and a substrate, wherein the substrate is selected from selenite, selenate, tellurate, nitrite, nitrate, arsenate, a compound that comprises Sb(V), and a combination thereof.

11. The composition of claim 10 further comprising a molecule that is oxidized in conjunction with the reduction of the substrate.

12. The composition of claim 11 wherein the molecule is an organic molecule.

13. The composition of claim 11 wherein the molecule is selected from lactate, pyruvate, fumarate, formate, casamino acids, yeast extract, and a combination thereof.

14. The composition of claim 11 wherein the substrate is a compound that comprises Sb(V), and the molecule has a standard midpoint reduction potential (E°′) of less than +94 mV.

15. The composition of claim 10 wherein the compound comprising Sb(V) is Sb(OH)₆⁻.

16. The composition of claim 10 further comprising an environmental sample, wherein the environmental sample comprises selenite, selenate, tellurate, nitrite, nitrate, arsenate, a compound that comprises Sb(V), or a combination thereof.

17. The composition of claim 16 wherein the environmental sample comprises soil, groundwater, surface water, wastewater, sediment, or a combination thereof.

18. A method comprising:
culturing the microbe of claim 1 in a composition comprising a substrate that is reduced by the microbe under suitable conditions.

19. The method of claim 18 wherein the substrate is a compound comprising Sb(V).

20. The method of claim 19 wherein the compound comprising Sb(V) is Sb(OH)₆⁻, and the reduction produces Sb₂O₃, wherein the Sb₂O₃ is sénarmontite, valentinite, or a combination thereof.

21. The method of claim 20 further comprising isolating the sénarmontite, the valentinite, or the combination thereof.

22. The method of claim 18 wherein the substrate is selected from selenite, selenate, tellurate, nitrite, nitrate, arsenate, and a combination thereof.

23. The method of claim 18 wherein the composition comprises an environmental sample.

24. The method of claim 23 wherein the environmental sample comprises soil, groundwater, surface water, sediment, or a combination thereof.

25. The method of claim 18 wherein the composition comprises a waste stream.

26. A method for making crystalline Sb₂O₃ comprising:
culturing the microbe of claim 1 in a composition, wherein the composition comprises a compound that comprises Sb(V), wherein the reduction of the Sb(V) results in Sb₂O₃, wherein the Sb₂O₃ is sénarmontite, valentinite, or a combination thereof, and wherein the culturing is under conditions suitable for the precipitation of the Sb₂O₃.

27. The method of claim 26 further comprising isolating the sénarmontite, the valentinite, or the combination thereof.

28. A method comprising:
culturing the microbe of claim 1 in a sample comprising a soluble contaminant that is reduced by the microbe under suitable conditions, wherein the soluble contaminant is selected from selenite, selenate, tellurate, nitrite, nitrate, arsenate, a compound that comprises Sb(V), and a combination thereof, wherein the reduction converts the soluble contaminant to an insoluble contaminant.

29. The method of claim 28 wherein the sample comprises an environmental sample.

30. The method of claim 29 wherein the environmental sample comprises soil, groundwater, surface water, waste water, sediment, or a combination thereof.

31. The method of claim 28 wherein the sample comprises a waste stream.

32. A method comprising:
adding the microbe of claim 1 to an environment comprising a soluble contaminant that is reduced by the microbe under suitable conditions, wherein the soluble contaminant is selected from selenite, selenate, tellurate, nitrite, nitrate, arsenate, a compound that comprises Sb(V), and a combination thereof, wherein the environment is anoxic, and wherein the reduction converts the soluble contaminant to an insoluble contaminant.

33. The method of claim 32 wherein the environment comprises soil, groundwater, surface water, waste water, sediment, or a combination thereof.

34. A method of isolating a microbe that reduces Sb(OH)₆⁻ to produce antimony trioxide under suitable conditions, comprising:
culturing the microbe in a composition comprising Sb(OH)₆⁻ and reduced sulfur, wherein the concentration of reduced sulfur is no greater than 200 μM.
